(19) United States
(12) Patent Application Publication
(10) Pub. No.: US 2012/0039926 A1
(43) Pub. Date: Feb. 16, 2012

(54) Compositions and Methods for Modulating Immunogenic Responses by Activating Dendritic Cells

(75) Inventors: Kenneth S. Rosenthal, Akron, OH (US); Daniel H. Zimmerman, Bethesda, MD (US); Patricia R. Taylor, Medina, OH (US)

(73) Assignees: CEL-SCI CORPORATION, Vienna, VA (US); NORTHEASTERN OHIO UNIVERSITIES COLLEGE OF MEDICINE, Rootstown, OH (US)

(21) Appl. No.: 13/264,546

(22) PCT Filed: Apr. 14, 2010
(86) PCT No.: PCT/US10/31054

§ 371 Date: Nov. 3, 2011

Related U.S. Application Data
(60) Provisional application No. 61/169,034, filed on Apr. 14, 2009.
(60) Provisional application No. 61/298,536, filed on Jan. 26, 2010.

(51) Int. Cl. A61K 39/385; A61P 35/00; A61P 31/00; A61P 37/02; A61P 37/08; A61P 37/04; A61P 31/04; A61P 31/12; A61P 31/10; A61P 33/02; A61P 33/00; A61P 31/18; A61P 31/22; A61P 31/16; C12N 5/0784

(52) U.S. Cl. 424/194.1; 435/377; 424/193.1

(57) ABSTRACT

The conjugated peptide constructs described herein can be used to induce production of dendritic cells that generate cytokines. The vaccine-bearing dendritic cells can be administered to induce T cell mediated immune modulating responses.

### CROSS-REFERENCE TO RELATED APPLICATIONS

This application claims the benefit of U.S. Provisional Application Ser. No. 61/169,034, filed Apr. 14, 2009, and Ser. No. 61/298,536 filed Jan. 26, 2010, disclosures of which are expressly incorporated herein by reference.

### STATEMENT REGARDING FEDERALLY SPONSORED RESEARCH

This invention was made with government support under Grant Nos. #R01 CA100163/CA and UL1RR024989/RR/NCRR, awarded by the National Institutes of Health. The government has certain rights in the invention.

### SEQUENCE LISTING

The instant application contains a Sequence Listing which has been submitted via EFS-Web and is hereby incorporated by reference in its entirety. The ASCII copy, created Apr. 14, 2010, is named 3358_{—}50877_SEQ_LIST_NEOUCOM.txt (1,111 bytes).

### TECHNICAL FIELD AND INDUSTRIAL APPLICABILITY OF THE INVENTION

This invention is directed, in part, to compositions and methods for conferring protection against autoimmune diseases, such as, for example, myocarditis, autoimmune, thyroid disease, rheumatoid arthritis, allergic diseases, asthma, host-versus graft, graft-versus-host disease, cancer, and chronic infections such as AIDS, malaria, herpes, hepatitis and tuberculosis.

The present invention is also directed, in part, to compositions and methods for activating and promoting the maturation of dendritic cell precursors (immature dendritic cells, or “iDCs”) or monocytes into dendritic cells (DC) and eliciting a favorable cytokine profile.

The present invention is also directed, in part, to compositions and methods for delivering minimal amounts of a vaccine of such DCs to elicit prophylactic or therapeutic responses.

The present invention is also directed, in part, to compositions and methods for modulating chronic inflammatory diseases such as those initiated by obesity or hypercholesterolemia. In such embodiments, the compositions need not be antigen specific, but rather a consequence of the IL12 producing cell.

The present invention is also directed to a method for treating cancer and chronic infections such as AIDS, cytomegalovirus, malaria, shingles, hepatitis and tuberculosis or inhibiting development of autoimmune diseases, asthma, allergy, and preventing tissue transplantation rejection and to conjugated peptides and compositions which may be used to carry out said method.

### BACKGROUND OF THE INVENTION

Peptide vaccines offer the advantage of a well defined immunogen that often ensure the generation of a safe and appropriate response in the vaccinated subjects. Due to their small size, these peptides are usually insufficient to induce an immune response by themselves. Rather, peptides have been attached to protein carriers such in order to become an immunogen. However, presentation of the peptide epitope in this manner is not optimal because this usually results in the development of a Th2 type of response or the immune responses that are elicited by the carrier protein.

Several technologies have been developed to convert epitope-bearing peptides into immunogens for vaccines, including, for example: attachment to Toll Like Receptor ligands, acylation of the peptide, attachment to Pan DR helper epitopes (PADRE) PaDre™; and li-key approaches.

### SUMMARY OF THE INVENTION

It has now been discovered by the present inventors that the L.E.A.P.S.™ constructs are necessary and sufficient to activate and promote the maturation of dendritic cell precursors (iDCs) into mature DCs and direct these DCs to elicit a desirable cytokine profile.

It has also been discovered by the present inventors that a subject's own iDCs cells from bone marrow (BM) can be activated and matured, also eliciting a favorable cytokine profile. It has also been discovered by the present inventors that a subject's own blood-derived monocytes can be activated and matured, also eliciting a favorable cytokine profile. The cytokine profile can either initiate, modulate, redirect or inhibit an immune response. That is, there can be specific cell activation (whether T helper cells, T suppressor cells or other T cells) with an antigenic peptide. Alternatively, there can be inhibition/suppression/modulation of the immune response in an antigen specific manner.

The ability to modulate (e.g., markedly increase, decrease, redirect or completely retard), in a patient-specific and antigen specific manner, a desired immune response outcome, while substantially maintaining the remainder of the immune response intact, is achieved through the methods and the conjugated peptide constructs of this invention.

Also, the ability to modulate (e.g., markedly increase, decrease, redirect or completely retard), in a patient-specific manner, a desired immune response outcome, while substantially maintaining the remainder of the immune response intact, is achieved through the methods and the conjugated peptide constructs of this invention. For example, the vaccine compositions described herein can be used to induce dendritic cell generated cytokines.

This invention provides a new DC and T cell modulation platform technology designed to synthesize novel peptide constructs that modify both cellular and humoral immune responses in a subject by using the subject's own immune system, without need for adjuvants.

Accordingly, it would be highly desirable to provide an immune therapy which would be effective to prevent initial infection as well as a treatment for individuals who suffer from chronic diseases, including infectious, autoimmune, chronic infection, allergy and cancer, or to immunomodulate undesirable immune responses, including graft vs host disease, without causing undesirable systemic and generic antigen-non-specific effects to the immune system, as would be caused by systemic treatment with an antagonist of an immune component.

Various objects and advantages of this invention will become apparent to those skilled in the art from the following detailed description of the preferred embodiment, when read in light of the accompanying drawings.

### BRIEF DESCRIPTION OF THE DRAWINGS

FIGS. 1A-1B. Survey of cytokine production following immunization of mice with J, JgD or JH.

FIG. 1A. C57BL/6 (n=3) or A/J (n=3) female mice were immunized with Seppic ISA51 or JgD in emulsion with the adjuvant. Sera were collected and pooled with the same volume of sera from each mouse in the group on days 3, 10, and 24 for each set of mice and evaluated on RayBiotech® mouse antibody microarrays. Duplicate spots for each cytokine per microarray were quantified by densitometry. Differences in results between C57BL/6 and A/j mice were not significant and means were determined for the cytokine values from both strains of mice, normalized to values for VEGF, and presented as a ratio to the values for the Seppic control. The highest standard deviation per cytokine was 0.04. The data is presented as the mean of values for both strain of mice.

FIG. 1B. A/J mice were immunized with JgD, JH or J and sera analyzed. Uncorrected p-values from a 2-way nested ANOVA comparing treatment and day post treatment are presented for immunized mice contrasted to adjuvant treated mice. Dark outlined boxes indicate significant results after the sequential Bonferroni correction for multiple contrasts.

FIGS. 2A-2D. Selected comparisons of serum cytokine levels following immunization of A/J mice with JgD, JH or J peptides in Seppic adjuvant. Values presented as a ratio to the values for the Seppic control for: IL-12p70 (FIG. 2A); IL-12p40 (FIG. 2B); IFN-γ (FIG. 2C); and, IL-10 (FIG. 2D) are plotted with respect to days after immunization.

FIGS. 3A-3D. Response of C57BL/6 bone marrow cells to JgD, J, gD, or JH immunogen treatments. JgD, J, gD, or JH were added to the BM cell suspensions and incubated for 48 hrs. FIG. 3A) Cells from 3 mice were then stained with PE-anti-CD11c; FIG. 3B) PE-anti-CD86 and flow cytometry was performed on cells within the suspension with light scatter parameters of monocytes. The X-mean for each evaluation indicates extent of antigen expression.

FIG. 3C-3D. In a separate experiment, BM cells from 5 C57BL/6 mice, FAC sorted to remove CD3+ cells, were untreated or treated with JgD or JH and the cell suspensions were incubated for 48 hours. Intracellular IL-12p70 and extracellular CD8 were evaluated on the entire sorted BM cell population. Immunofluorescence was analyzed and compared to isotype controls. The Table represents the percent of positive cells for each quadrant.

FIGS. 4A-4B. Response of purified iDCs to JgD immunogen treatment. iDCs from C57BL/6 mice (n=5) were pooled and untreated or treated with 3.25, 7.25, or 14.5 micromoles of JgD. FIG. 4A) After 48 hrs, cells were microscopically examined for morphological changes; and, FIG. 4B) a direct IL-12p70 ELISA was performed in triplicate on the supernatants from cell suspensions of two independent trials, values were averaged, and error bars indicate standard deviation between ELISA values. Values were significantly different for the different dose amounts (p<0.05) as per ANOVA.

FIG. 5. Cytokine response of co-cultures of immunonaïve splenocytes with JgD-BM or JH-BM cells. BM cells from 3 C57BL/6 mice were pooled and aliquots (2×10⁶) were untreated or treated with 14.5 micromoles of JH or JgD and incubated for 48 hrs on two separate occasions. The BM cells were washed and then added to 2×10⁷ splenocytes (pooled from 3 mice), and incubated for 48 hrs. Supernatants were removed and evaluated by RayBiotech® mouse antibody microarrays. “Spots” were quantified by densitometry, means and standard deviation were determined, normalized to total array values to allow comparison, and presented as a ratio of values for treated BM cells to untreated BM cells. Error bars indicate the standard deviation between two separate experiments.

FIGS. 6A-6B. Treatment with JgD or JH promotes maturation of human monocytes into dendritic cells. Monocytes obtained by leukapheresis of blood and purified by elutriation were cultured in serum free media supplemented with human GMCSF 50 ng/ml and IL4 (500 U/ml) or 24 h. The cells were then treated with 14.5 μmol of JgD or JH and incubated for 3 days at 37° C.

FIG. 6A. Microscopic photographs of human monocytes show the phenotypic changes after treatment including dendrite formation and clustering of the cells.

FIG. 6B. Cells shown were fixed, stained with PE-anti-CD86 or PE-anti-DR and analyzed by flow cytometry.

FIG. 7. Survey of cytokine production following JgD or JH treatment. Human blood derived monocytes were treated with JgD or JH in two separate experiments. Spent media were collected three days post treatment, and evaluated by protein array (RayBio® Human Cytokine Antibody Array 3). Array results were quantitated by densitometry, and normalized to the summation values for each array to allow for comparative analysis of JgD or JH treated to untreated dendritic cell array results. The data shown are the mean scores for the fold increase or decrease to the untreated control for each of the 42 cytokines on the replicated arrays. The error bars represent the standard deviation between trials. Inset, human monocytes from different donors produced similar amounts of IL12p70 after being treated with JgD. Spent media was obtained from monocytes from donors 3, 5, and 8 after incubation with JgD in separate and repeated experiments, and analyzed, as discussed above. (*) Significant change in cytokine production from untreated cells.

FIG. 8. JgD treated human monocytes activate allogeneic T cells to produce IFNγ and IL2. Monocytes and T cells were obtained after elutriation of the human apheresis product. CD4⁺ T cells were further purified with T cell isolation columns. Monocytes harvested 24 h after treatment with JgD or HBSS were added to T cell cultures at a 1 DC: 10 T cell ratio. Spent media were collected six days after co-culture, and assayed by protein array as described above. (*) Significant change in cytokine production from untreated cells.

FIG. 9. Kaplan Meier survival curve for mice vaccinated with either the JgD-DC or untreated BM receiving lethal challenge with herpes simplex virus type 1 by zosteriform challenge.

FIG. 10. Reduction in symptoms of mice (see FIG. 9) treated with JgD-DC vaccine, as compared with: No treatment; Untreated BM vaccine; J-BM vaccine; or JH-DC vaccine.

### DETAILED DESCRIPTION OF THE PREFERRED EMBODIMENT

The present invention can be practiced with a class of immunologically active and diagnostic peptide constructs that are obtained by joining one or more T cell or immune cell binding ligands with an antigenic peptide. These peptide fragments and/or constructs are described, for example: Zimmerman et al. U.S. Pat. No. 5,652,342; Zimmerman et al. U.S. Pat. No. 6,093,400; Zimmerman et al. U.S. Pat. No. 6,096,315; Zimmerman et al. U.S. Pat. No. 6,111,068; Zimmerman et al. U.S. Pat. No. 6,258,945; Zimmerman et al. U.S. Pat. No. 6,268,472; Zimmerman US Pub. No. 2006/0134216; and Zimmerman US Pub. No. 2007/0003542, the entire disclosures of which are expressly incorporated herein, in their entireties.

Specific classes of peptide constructs (e.g., “P₁-x-P₂” peptide constructs, also called the “L.E.A.P.S.™” technology herein) have been developed for a number of specific infectious and immunological disorders, including, for example: HIV-1 (e.g., Zimmerman et al. U.S. Pat. No. 6,103,239; Zimmerman et al. U.S. Pat. No. 6,287,565); HSV (e.g., Zimmerman et al. U.S. Pat. No. 6,572,860); and, autoimmune diseases, host v graft diseases (e.g., Zimmerman U.S. Pat. No. 6,951,647; Zimmerman U.S. Pat. No. 6,995,237; Talor U.S. Pat. No. 7,199,216; Zimmerman U.S. Pat. No. 7,256,254; Zimmerman US Pub. No. 2006/0257420; Talor US Pub. No. 2007/0128698); the disclosures of which are expressly incorporated herein in their entireties.

In addition, the co-pending applications are expressly incorporated herein by reference: U.S. Ser. No. 61/036,566 filed Mar. 14, 2008; U.S. Ser. No. 61/61/100,383 filed Sep. 26, 2008, both of which were incorporated into PCT/US application filed Mar. 14, 2009 and U.S. Ser. No. 11/443,314 filed May 31, 2006; and, provisional patent applications on swine influenza H1N1 L.E.A.P.S.™ conjugates 61/185,565 filed Jun. 9, 2009 and 61/234,966 filed Aug. 28, 2009.

One technique that is useful for modulating T cell immunological responses to a wide range of antigenic peptides by targeting and activating dendritic cells is be referred hereinto as Ligand Epitope Antigen Presentation System, or L.E.A.P.S.™. The L.E.A.P.S.™ technology provides conjugated peptide immunogens (constructs) that modulate both cellular and humoral responses to treat and/or prevent major diseases, such as HIV infection, herpes simplex virus (HSV) infection, tuberculosis, and autoimmune diseases, such as rheumatoid arthritis, insulin dependent diabetes, multiple sclerosis and the like.

The L.E.A.P.S.™ constructs are conjugates of two peptides which are linked together covalently, and can be generally described as having the formula: P₁-x-P₂, where “P₁” represents an immunomodulatory peptide which is a portion of an immunoprotein capable of promoting binding to a class or subclass of DC or T cells and which is capable of directing a subsequent immune response to the peptide P₁ to a Th1 or other immune response; “P₂” represents a specific antigenic peptide; and “x” represents a covalent bond or a divalent peptide linking group, which may be cleavable or non-cleavable. One peptide (hereinafter may be referred to as Peptide P₂) of the conjugate is an antigen-specific epitope which will bind to the T cell receptor upon recognition. The other peptide of the conjugate, P₁, is an immunomodulating cell binding ligand (ICBL) or T cell binding ligand (hereinafter may be referred to as ICBL, Peptide P₁,) derived from molecules with a known activity, such as, for example, β-2 microglobulin, IL-1, IL-2, or nonpolymorphic MHC regions, and which will engage other sites on the T cells or other immune cells to ultimately promote activation of a particular set or subset of T cells. A more detailed discussion of the L.E.A.P.S.™ peptide constructs can be found in the above-mentioned U.S. Pat. No. 5,652,342.

The L.E.A.P.S.™ constructs allow for the preferential presentation of antigen(s) (peptide sequences) to antigen presenting cells, lymphocytes (T and B cells), dendritic cells, and other cells of the immune system. The antigen presentation is directed in such a way as to affect immune response outcome and determine, with some certainty, the type of immune response outcome, humoral plus cellular (Th1 type of response), or only humoral (Th2 type of response). Other types of response (Treg) may also be possible depending upon the L.E.A.P.S.™ construct. Thus, with the use of certain combinations of appropriate T cell binding peptide molecules together with the appropriate antigen, or the pathogenic molecule(s) of a complex antigen, forming the L.E.A.P.S.™ construct, a cellular, antibody, or a mixed immune response can be induced or modulated by administration of the L.E.A.P.S.™ construct.

In another example, as set forth in U.S. Pat. No. 7,199,216, there is described the use of modified L.E.A.PS.™ constructs for the prevention of graft-versus-host (GvH) rejection using a host antigen that is mixed with donor bone marrow cells prior to infusion.

In another example, the U.S. Pat. No. 6,572,860 describes an artificial gene for a L.E.A.P.S.™ construct, using various different eukaryotic cell expression vector devices to allow translation and production of the L.E.A.P.S.™ constructs as a “mini protein.”

In connection with the L.E.A.P.S.™ technology as previously described in the aforementioned U.S. Pat. No. 5,652,342, it is believed that the antigen portion of these constructs interact in a direct manner, primarily to T cells, utilizing the presence of various cell surface molecules and receptors on the T cell. The antigen (in conjunction with the L.E.A.P.S.™ construct) interacts with the antigen-specific T cell receptor on the T cell surface, providing the primary signal—the first of two signals required for T cell activation. The L.E.A.P.S.™ construct, itself derived from homologous sequences of MHC (HLA) class I and Class II molecules, among others (see, e.g., U.S. Pat. No. 5,652,342) interacts with accessory molecules on the same T cell—providing the secondary signal required for T cell activation.

In contrast, according to the present invention, the L.E.A.P.S.™ peptide construct is bound to dendritic cells (DCs) to such an extent that the autoimmune associated antigenic peptides (or asthma, allergy or transplantation rejection antigens) are still able to interact with the T cell receptor and to provide the primary signal to the T cell, while simultaneously preventing the secondary signal required for T cell activation.

Thus, the inventors herein have now discovered a dramatically different method which uses a subject's own immune response. In one embodiment, the method can generally include:

i) extracting dendritic cell (DC) precursor cells from a subject and isolating away from other body tissues; and

ii) culturing the isolated DC precursor cells from the subject with a L.E.A.P.S.™-type peptide construct (or, in certain other embodiments, a similarly acting peptide construct) in order to activate, mature, and direct the character of any resulting mature DCs.

In certain embodiments, the method includes reinfusion of the isolated mature DCs back into the same subject, such that these activated mature DCs will interact with T cells and B cells, (and possibly others, such as macrophages) of the subject and provide a specific, focused (via the antigenic peptide component of the L.E.A.P.S.™ peptide construct) and directed immunomodulation of T cells, through the DC derived cytokines that are activated by the components of the L.E.A.P.S.™ peptide construct.

In certain embodiments, DC precursor cells may be incubated with L.E.A.P.S.™ peptide constructs without or with GM-CSF (granulocyte monocyte colony stimulating factor) with or without IL4 (interleukin 4).

The present invention is also based, in part, on the inventor discovery that the L.E.A.P.S.™ peptide constructs can be used ex vivo to produce a desired immune response in cells taken from a subject.

For ease of explanation, the L.E.A.P.S.™ peptide constructs are sometimes described herein and are represented by the formula P₁-x-P₂. Non-limiting examples of useful “P₁”, “P₂” and “x” groups are described in the above-mentioned references which have been fully incorporated herein by reference.

In certain embodiments, P₁ is a immune modulating peptide (ICBL); P₂ is a peptide which binds to an antigen receptor on a set or subset of T cells which binds to monocytes or bone marrow cells, promotes maturation to DCs and resultant DCs to initiate a directed immune response and presents P₂ to a T cell receptor which causes the set or subset of T cells to which the peptide P₂ is bound to specifically modulate an immune response in the subject; and x is a direct bond or a linker moiety for covalently bonding P₁ and P₂

For the peptides disclosed above and below and as employed in the experimentation described herein, the amino acid sequences thereof, are set forth by the single identification letter or three-letter identification symbol as follows: Name, three-letter, One-letter Amino Acid abbreviation symbol: Alanine, Ala, A; Arginine, Arg, R; Asparagine, Asn, N, Aspartic Acid, Asp, D; Cysteine, Cys, C; Glutamine, Gln, Q; Glutamic Acid, Glu, E; Glycine, Gly, G; Histidine, His, H; Isoleucine, Be, I; Leucine, Leu, L; Lysine, Lys, K; Methionine, Met, M; Phenylalanine, Phe, F; Proline, Pro, P; Serine, Ser, S; Threonine, Thr, T; Tryptophan, Trp, W; Tyrosine, Tyr, Y; and Valine, Val, V.

It should be understood that in any of the amino acid sequences specified herein variations of specific amino acids which do not adversely effect the desired biological activity are contemplated and fall within the scope of the invention. Although the regions of interest of the preferred antigenic peptides are highly conserved, natural and spontaneously occurring amino acid variations are specifically contemplated. In some cases, it may be advantageous to use mixtures of peptides, the sequences of which, within the guidelines given above, and discussed in more detail below, correspond to two or more natural and spontaneously occurring variants.

Still further, as well recognized in the art, it is often advantageous to make specific amino acid substitutions in order, for example, to stabilize, prevent random polymerization, cyclization, and cross-linking, solubilize, provide specific binding sites or for purpose of introducing radioactive or radioisotope, toxic drugs or fluorescent tagging of the peptide. Tagging with other types of identifying labels, as well known in the art, such as, for example, toxins and drugs, may also advantageously be included in or with the conjugated peptides of this invention. Such “designed” amino acid sequences are also within the scope of the antigenic peptides of this invention.

In addition, it is also recognized that the amino acids at the N-terminal and C-terminal may be present as the free acid (amino or carboxyl groups) or as the salts, esters, ethers, or amides thereof. In particular amide end groups at the C-terminal and acetylation, e.g., myristyl, etc. at the N- or C-terminal, are often useful without effecting the immunological properties of the peptide.

The peptides P₁ and P₂ (as hereinafter defined) of the conjugated polypeptides of the present invention can be prepared by conventional processes for synthesizing proteins, such as, for example, solid phase peptide synthesis, as described by Merrifield, R. B., 1963, J. of Am. Chem. Soc., 85:2149-2154.

It is also within the scope of the invention and within the skill in the art to produce the novel conjugated peptides or the peptide components thereof by genetic engineering technology.

In a first aspect, there is provided herein a dendritic cell (DC) and T cell modulation platform technology that uses a peptide construct that modifies cellular and/or humoral immune responses of a subject by reacting with a subject's own immune system and/or cells derived from the subject's immune system, without need for adjuvants or “non-self” antigens, or cells compatible with an individual's immune system (e.g., MHC compatible).

In another aspect, there is provided herein a method for producing a mature dendritic cell (DC) population, which method comprises: contacting at least one precursor of dendritic cells (i.e., “immature dendritic cells” or “iDCs”) with an effective amount of a peptide construct having the formula P₁-x-P₂ under culture conditions suitable for maturation of the iDCs into a mature dendritic cell (DC) population.

Further, in certain embodiments, the mature DC population produces an immunomodulatory response with an increased amount of interleukin 12 (IL-12), as compared to cells not contacted with the peptide construct.

In another aspect, there is provided herein a composition for activating T cells, comprising: a dendritic cell population matured using an effective amount of a peptide construct having the formula P₁-X-P₂ under culture conditions suitable for maturation of the iDCs into a mature dendritic cell (DC) population.

Further, in certain embodiments, the mature DC population produces an immunomodulatory response with an increased amount of interleukin 12 (IL-12) compared to cells not contacted with the peptide construct. While not wishing to be bound by theory, the inventors' herein believe that the P₁-x-P₂ bound to the DC cell surface binds to T cells through the P₂ antigenic peptide and other DC T cell receptor interactions and modulates the T cell activity through these cell-cell interactions and through the cytokines that the DC produces.

In another aspect, there is provided herein an autologous method for modulating a response (e.g., one or more of activation, differentiation or suppression) to an immunogen in a subject in need thereof, comprising: combining extracted iDCs cells with a LEAPS peptide construct ex vivo to form a mixture, and administering the mixture to the subject. In certain embodiments, the mixture can be soon thereafter be directly administered to the subject. In certain other embodiments, the mixture can be administered to the subject ex vivo after incubation in cell culture.

In another aspect, there is provided herein compositions and methods for modulating chronic inflammatory diseases such as those initiated by obesity or hypercholesterolemia. In such embodiments, the compositions need not be antigen specific, but rather a consequence of the IL12 producing cell.

In another aspect, there is provided herein an autologous method for modulating a response to an immunogen in a subject in need thereof, comprising: obtaining a cell population of iDCs from a subject; differentiating the iDCs into mature DCs in the presence of a peptide construct; and introducing the “mature DC-(P₁-x-P₂) complex” vaccine back into the subject.

In another aspect, there is provided herein an autologous method for modulating a response to an immunogen in a subject in need thereof, comprising: treating isolated iDCs from blood derived monocytes and/or bone marrow taken from a subject with a peptide construct having the formula P₁-x-P₂ to induce maturation of the iDCs into mature dendritic cells (DC); harvesting a supply of the mature DCs; and, administering (optionally, with a supplementary immunomodulator) an effective amount of the harvested mature DCs to the subject. It is to be understood that, in certain embodiments, the supplementary immunomodulators can be, for example, immune activators, cytokines and/or chemokines.

However, it should be understood that the present methods and compositions described herein provides a clear advantage in that the DCs are functional after being washed of free L.E.A.P.S.™ peptide. That is, all of the “mature DCs—L.E.A.P.S.™ peptide” vaccine administered to the subject is bound to the cells. As such, the amount of peptide administered to the subject is minimized and cannot affect other cells in the subject's body, thereby minimizing the potential for toxicity and/or unpredictable actions.

In another aspect, there is provided herein an autologous method of inducing a systemic antigen specific immune response in a subject, comprising: isolating immature dendritic cells (iDCs) from blood derived monocytes and/or bone marrow taken from the subject; treating the isolated iDCs with a peptide construct having the formula P₁-x-P₂ to induce maturation of the iDCs into mature dendritic cells (DCs); harvesting a supply of the mature DCs; and, administering (optionally, with a supplementary immunomodulator) an effective amount of the harvested mature DCs to the subject.

In another aspect, there is provided herein an autologous method of inducing a systemic antigen specific immune response in a subject, comprising: isolating precursors of dendritic cells (iDCs) from blood derived monocytes and/or bone marrow taken from the subject; treating the isolated iDCs with a peptide construct having the formula P₁-x-P₂ to induce maturation of the iDCs into mature dendritic cells (DCs); harvesting a supply of the mature DCs; mixing the DCs with autologous T cells and, administering (optionally, with an adjuvant) an effective amount of the mixture of cells to the subject. In certain embodiments, when monocytes are isolated from the subject, lymphocytes can also be obtained.

After the DCs are obtained from the monocytes, the isolated DCs can be mixed with the T cells that were obtained (e.g., frozen for later use), perform the activation and expansion of T cells ex vivo and then reinfuse the mixture into the subject. Also, it is to be understood that the methods described herein are useful with fresh or revitalized, previously frozen iDCs.

In still other embodiments, the iDCs can be treated with a mixture of L.E.A.P.S.™ peptides of formula P₁-x-P₂, in which P₂ could be varied and/or may come from the same protein or from another protein involved in eliciting therapy.

In still other embodiments, subjects can be treated with a mixture of DCs treated separately with peptides P₁-x-P₂, differing in P₂ in which P₂ may come from the same protein or from another protein involved in eliciting therapy.

In another aspect, there is provided herein isolated mature dendritic cells (DCs) that are capable of producing both an immunomodulatory response and interleukin 12 (IL-12), where the DCs are prepared by maturation of iDCs with a peptide construct of formula P₁-x-P₂ under conditions suitable for the maturation of the dendritic cells.

In certain embodiments, the peptide construct is capable of directly inducing a dendritic cell immune response.

In certain embodiments, the cultured DCs are characterized by up-regulation of at least one of: CD11c, CD86, MHC class I or MHC class II cell surface marker.

In certain embodiments, the mature DCs are capable of producing a desired cytokine profile.

In certain embodiments, the mature DCs produce IL-12.

In another aspect, there is provided herein a pharmaceutical composition comprising an effective amount of the mature DCs produced by any of the methods described herein.

In certain embodiments, the pharmaceutical composition is useful in eliciting an immunotherapeutic response to an infection or neoplastic disease, whereby administration to the subject elicits a cell-mediated response, against the infection or neoplastic disease.

In certain embodiments, the pharmaceutical composition is useful for the manufacture of a medicament for use in eliciting an immunotherapeutic response to an infection or neoplastic disease, whereby the administration to the subject elicits a cell-mediated response, against the infection or neoplastic disease.

In certain embodiments, composition is administered directly into or around a tumor, infected tissue or organ presented by the subject, or into the draining lymph node or peritoneum of the patient.

In another aspect, there is provided herein a method of treating an infection or neoplastic disease or aberrant cell population by administering a therapeutically effective amount of the pharmaceutical composition to the subject in need thereof.

In another aspect, there is provided herein an autologous method of inducing proliferation of a cell population containing mature dendritic cells in a subject. The method generally comprises: contacting blood derived monocytes and/or bone marrow cells of the subject with a immunomodulatory peptide construct having the formula P₁-X-P₂. In another aspect, there is provided herein a method of treating at least one cell proliferation disorder, the method comprising: administering a therapeutically effective amount of a pharmaceutically acceptable composition comprising the harvested cells.

In another aspect, there is provided herein a method for treating other cellular disorder, including, but not limited to: excessive (hyper-) or reduced (hypo-) responses such as hormone or other protein production or other metabolic responses. In certain embodiments, the cell hypersecretion is excessive hormone secretion disorder, such as for example, disorders of: adrenal glands, ovaries, testes, thyroid, pituitary glands, pancreas and the like.

In a particular embodiment, the cell hypersecretion is an ecotopic hormone secretion disorder. Also, in certain embodiments, the method is useful to treat cell proliferation disorder such as, but not limited to: autoimmune diseases, graft v host (GvH), host vs graft (HvG) diseases, and/or acute, latent-recurring and chronic infectious diseases.

In certain embodiments, the cell proliferation disorder is cancer.

In another aspect, there is provided herein a method for treating or preventing cancer, infectious diseases, autoimmune disease, asthma, allergy, atopic dermatitis, psoriasis, and transplantation rejection, by administering to a subject in need thereof a therapeutically effective amount of the compositions as described herein.

In another aspect, there is provided herein a method of inducing an adaptive immune response in a subject to a target antigen, the method comprising: administering to the subject a peptide construct having the formula P₁-x-P₂ in an amount effective to induce the response.

In another aspect, there is provided herein use of autologous mature DCs formed by the methods described herein in the manufacture of a medicament for the induction of an adaptive immune response.

In another aspect, there is provided herein compositions for initiating an immune response, the composition comprising: an autologous antigen-presenting mature dendritic cell (DC) produced by any of the methods described herein.

In another aspect, there is provided herein a method of controlling an immunodeficiency viral load of a subject, the method comprising the steps of administering the composition at a dosage and for a time sufficient to reduce the immunodeficiency viral load.

In another aspect, there is provided herein a method of inducing an immune response in a subject, the method comprising administering the composition to the subject at a dosage and for a time sufficient to induce protective immunity against subsequent infection.

In another aspect, there is provided herein a method of inducing protection and preventing or minimizing development of an inappropriate cytokine response (e.g., cytokine storm) by an infection.

In another aspect, there is provided herein a method of inducing a CD8 T cell response to an antigenic peptide in a subject in need thereof, the method comprising: culturing immature dendritic cells (iDCs) from the subject in the presence of a peptide construct having the formula P₁-x-P₂, to provide cultured mature dendritic cells DCs which express IL-12; and subsequently reintroducing the mature DCs to the same patient.

In certain embodiments, the cultured DCs are characterized by up-regulation of at least one of the following: CD11c CD86, MHC class I or MHC class II cell surface marker.

In certain embodiments, the subject is a human.

In another aspect, there is provided herein a method of inducing a CD8 T cell response in a subject in need thereof, the method comprising: contacting precursors of dendritic cells obtained from the subject with a peptide construct having the formula P₁-x-P₂ to generate mature dendritic cells (DCs) capable of producing a desired cytokine profile. In certain embodiments, the mature DCs produce interleukin 12 (IL-12).

In another aspect, there is provided herein a method for inducing and/or inhibiting suppressor/regulatory T lymphocytes and/or inflammatory T lymphocytes (interleukin 17 releasing Th17 T cells) in a subject in need thereof, the method comprising: using mature autologous dendritic cells expressing an antigen to the peptide construct of the formula P₁-x-P₂.

In another aspect, there is provided herein a method for producing a mature dendritic cell (DC) population, the method comprising: providing precursors of dendritic cells from a subject; and contacting the precursors of dendritic cells (iDCs) with an effective amount of a peptide construct having the formula P₁-x-P₂ under culture conditions suitable for maturation of the iDCs to form a mature dendritic cell (DC) population; wherein the mature DC population produces an immunomodulatory response and an increased amount of interleukin 12 (IL-12) compared to an iDC population not contacted with the peptide construct to generate a Th1 response to antigens or to immunomodulate an ongoing immune response.

In another aspect, there is provided herein a method for producing an immune response in a subject, comprising: providing immature dendritic cells (iDCs); contacting the iDCs with effective amounts of a peptide construct having the formula P₁-x-P₂ under culture conditions suitable for maturation of the iDCs to form mature dendritic cells (DCs); and administering the mature DCs to the subject.

In another aspect, there is provided herein a method for producing a regulatory or suppressive response in a subject, the method comprising: providing precursors of dendritic cells (iDCs); contacting the iDCs with effective amounts of a peptide construct having the formula P₁-x-P₂ under culture conditions suitable for maturation of the iDCs to form mature dendritic cells (DCs); and administering the mature DCs to the subject.

The present invention also relates to pharmaceutically effective compositions containing a conjugated polypeptide, as described herein. In certain embodiments, the compositions are useful for eliciting a desired immune response in a human subject.

Similarly, the invention relates to the use of such conjugated polypeptide and the pharmaceutically effective composition containing the same for treating or preventing infection by administering to a human patient in need thereof, a therapeutically or prophylactively effective amount of the conjugated polypeptide, as defined herein.

The invention will now be described in further detail by way of the following explanations and Examples.

It is to be noted that, while the following examples, describe primarily the immune cell binding ligands (ICBLs) containing the sequence of Peptide J and the sequence for Peptide G, it is to be understood that other ICBL peptides may be conjugated to suitable peptides derived from disease causing organisms, and/or from antigenic peptides associated with a particular disease, disorder or condition, in place of the conjugated peptides in order to achieve similar results.

Similarly, for treatment of certain diseases, conditions or disorders, the antigenic peptide can be chosen from the particular antigenic peptides associated with, or causing, the particular disease, disorder or condition, such as described in the references incorporated herein, or any of the other copending applications, or any other of the myriad known antigenic peptides associated with disease or causing disease.

### EXAMPLES

The present invention, in one specific aspect thereof, provides a novel immunomodulatory complex effective for the treatment and/or prevention of a disease in a subject, comprising:

a pharmaceutically effective amount of a mature dendritic cell (DC) population having at least one peptide construct at least partially attached or bound to the surface of the dendritic cells, the peptide construct having a formula: P₁-x-P₂, where

“P₁” represents an immunomodulatory peptide which is a portion of an immunoprotein capable of promoting binding to a class or subclass of DC or T cells and which is capable of directing a subsequent immune response to the peptide P₁ to a Th1 or other immune response;

“P₂” represents a specific antigenic peptide; and

“x” represents a covalent bond or a divalent peptide linking group, which may be cleavable or non-cleavable.

The present invention, in one specific aspect thereof, provides a novel complex for activating T cells, comprising a dendritic cell (DC) population matured with an effective amount of a peptide construct having the formula P₁-x-P₂ under conditions suitable for maturation of precursors of dendritic cells (iDCs) to form the mature dendritic cells (DCs).

In certain embodiments, the peptide construct is capable of modifying cellular and/or humoral immune responses of a subject by reacting with the subject's own immune system and/or cells derived from the subject's immune system, without need for adjuvants or “non-self” antigens. In certain embodiments, wherein a population of the mature dendritic cells (DCs) produce an immunomodulating response and an increased amount of interleukin 12 (IL-12) compared to an iDC population not contacted with the peptide construct.

In certain embodiments, the complex is effective as an immunogen is a vaccine for the treatment or prevention of the disease.

In certain embodiments, the complex is capable of electing a cellular immune response when administered to the subject in need thereof.

In certain embodiments, the P₁ and P₂ are derived from different molecules.

In certain embodiments, the precursor, or immature, dendritic cells (iDCs) are derived from the subject.

In certain embodiments, the precursor, or immature, dendritic cells (iDCs) are derived from a donor compatible with the subject.

In certain embodiments, the P₂ is an antigenic peptide, or fragment thereof, associated with a disease selected from one or more of: an allergen, an autoimmune-related antigen, a transplantation autoimmune response, a tumor antigen, an acute, latent-recurring and/or chronic inflammatory response.

In certain embodiments, a causative agent of the disease to which the antigenic peptide is associated is one or more of: bacteria, viruses, fungi, protozoa, parasites and prions.

In certain embodiments, a disease related human protein or analogue from non-human sources to which the antigenic peptide is associated.

In certain embodiments, the complex is capable of initiating an antigen-specific immunomodulatory therapeutic response in the subject.

In certain embodiments, the complex is capable of initiating an antigen-specific immunomodulatory therapeutic response by activation of T cells of the subject.

In certain embodiments, the complex is configured such that the T cells are activated ex vivo.

In certain embodiments, the complex is capable of promoting a systemic modulation of immune and inflammatory responses in the subject sufficient to initiate a non-specific immunomodulatory therapeutic response to a chronic condition in the subject.

In certain embodiments, the complex is capable of producing interleukin-12 (IL-12).

In certain embodiments, the complex comprises two or more peptide constructs capable of stimulating the DCs individually before being administered to the subject.

In certain embodiments, the complex comprises precursor, or immature, dendritic cells (iDCs) that are derived from the subject.

In certain embodiments, the complex is substantially free of unbound peptide constructs.

In certain embodiments, the peptide construct comprises an immune cell binding ligand, termed “J”, an amino acid 38-50 from the β-2-microglobulin (DLLKNGERIEKVE) [SEQ ID NO:1] conjugated to a peptide from the N-terminus of HSV-1 glycoprotein “D” (SLKMADPNRFRGKDLP) [SEQ ID NO:2], amino acid 8-23) through a triglycine linker.

In certain embodiments, the peptide construct comprises an immune cell binding ligand, termed “J”, an amino acid 38-50 from the β-2-microglobulin (DLLKNGERIEKVE) [SEQ ID NO:1], conjugated to a HGP-30 peptide from the p17 HIV gag protein “H” (YSVHQRIDVKDTKEALEKIEEEQNKSKKKA) (aa 85-115)) [SEQ ID NO:3] through a triglycine linker.

The present invention, in one specific aspect thereof, provides a novel method for producing a mature dendritic cell (DC) population, comprising the step of: contacting precursor, or immature, dendritic cells (iDCs) with an effective amount of a peptide construct under conditions suitable for forming mature dendritic cells (DC), the peptide construct having a formula: P₁-x-P₂, where P₂ represents a specific antigenic peptide; P₁ represents an immunomodulatory peptide which is a portion of an immunoprotein capable of promoting binding to a class or subclass of DC or T cells and which is capable of directing a subsequent immune response to the peptide P₂ to a Th1 or other immune response; and x represents a covalent bond or a divalent peptide linking group, which may be cleavable or non-cleavable.

The present invention, in one specific aspect thereof, provides a novel method of reducing chronic inflammatory responses comprising an autologous mature IL12 producing DC produced by the method herein.

In certain embodiments, a population of mature DCs produces an immunomodulating response with an increased amount of interleukin 12 (IL-12) as compared to an iDC population not contacted with the peptide construct.

In certain embodiments, the precursor, or immature, dendritic cells (iDCs) comprise one or more of: blood derived monocytes and bone marrow cells.

In certain embodiments, the peptide construct is capable of directly inducing a dendritic cell immune response, wherein dendritic cell maturation is increased.

In certain embodiments, the mature DCs are characterized by up-regulation of at least one of: CD11c and CD86.

In certain embodiments, the mature DCs are capable of producing a desired cytokine profile.

In certain embodiments, the mature DCs produce interleukin 12 (IL-12).

In certain embodiments, wherein the mature DCs are administered directly into or around a tumor, infected tissue or organ presented by the subject, or into a draining lymph node or peritoneum of the subject.

In certain embodiments, the method includes inducing proliferation of a cell population containing mature dendritic cells (DCs) by contacting blood derived monocytes and/or bone marrow cells of the subject with the peptide construct.

In certain embodiments, the subject is a human.

The present invention, in one specific aspect thereof, provides a novel autologous method for modulating a response to an immunogen in a subject in need thereof, comprising:

i) combining precursor, or immature, dendritic cells (iDCs) extracted from the subject with a peptide construct having the formula P₁-x-P₂ to form a complex; and

ii) administering the complex to the subject.

In certain embodiments, the mixture is administered to the subject after the mixing step without any further incubation of the iDCs.

In certain embodiments, the mixture is administered to the subject after ex vivo incubation of the iDCs in cell culture.

The present invention, in one specific aspect thereof, provides a novel autologous method for modulating a response to an immunogen in a subject in need thereof, comprising:

i) differentiating precursor, or immature, dendritic cells (iDC) from a subject ex vivo into mature dendritic cells (DCs) in the presence of a peptide construct having the a formula: P₁-x-P₂; and

ii) introducing the mature DCs back into the subject.

The present invention, in one specific aspect thereof, provides a novel autologous method for modulating a response to an immunogen in a subject in need thereof, comprising:

i) treating isolated precursor, or immature, dendritic cells (iDCs) from blood derived monocytes and/or bone marrow taken from a subject with a peptide construct to induce maturation of the iDCs into mature dendritic cells (DC); the peptide construct having a formula: P₁-x-P₂; and,

ii) administering, optionally without any supplementary immunomodulators, an effective amount of the mature DCs to the subject.

The present invention, in one specific aspect thereof, provides a novel autologous method of inducing an antigen specific immune response in a subject, comprising:

i) treating precursor, or immature, dendritic cells (iDCs) from a subject with a peptide construct to induce maturation of the iDCs into mature dendritic cells (DCs); the peptide construct having a formula P₁-x-P₂; and,

ii) administering, optionally without any supplementary immunomodulators, an effective amount of the mature DCs to the subject.

The present invention, in one specific aspect thereof, provides a novel autologous method of inducing a systemic antigen non-specific immune response in a subject, comprising:

i) treating precursor, or immature, dendritic cells (iDCs) from blood derived monocytes and/or bone marrow taken from a subject with a peptide construct to induce maturation of the iDCs into mature dendritic cells (DCs); the peptide construct having a formula: P₁-x-P₂;

ii) mixing the mature DCs with autologous T cells to form a complex; and,

iii) administering, optionally without any adjuvant, an effective amount of the complex to the subject.

The present invention, in one specific aspect thereof, provides a novel isolated mature dendritic cell (DC) population, comprising DCs capable of producing an immunomodulatory response, the mature DCs being prepared by maturation of precursor, or immature, dendritic cells (iDCs) in the presence of a peptide construct under conditions suitable for the maturation of the dendritic cells, the peptide construct having a formula: P₁-x-P₂.

The present invention, in one specific aspect thereof, provides a pharmaceutical composition comprising an effective amount of the complex described herein.

The use of the pharmaceutical composition, for use in eliciting an immunotherapeutic response to an infection or neoplastic disease, whereby administration to the subject elicits a cell-mediated response, against the infection or neoplastic disease.

The present invention, in one specific aspect thereof, provides a novel use of the pharmaceutical composition, for the manufacture of a medicament for use in eliciting an immunotherapeutic response to an infection or neoplastic disease, whereby the administration to the subject elicits a cell-mediated response, against the infection or neoplastic disease.

The present invention, in one specific aspect thereof, provides a novel method of treating an infection or neoplastic disease, comprising: administering a therapeutically effective amount of the pharmaceutical composition, to a subject in need thereof.

The present invention, in one specific aspect thereof, provides a novel method of treating a cell proliferation disorder comprising the step of: administering a therapeutically effective amount of a pharmaceutically acceptable composition, to a subject in need thereof.

The present invention, in one specific aspect thereof, provides a novel method for treating a cellular disorders including, but not limited to: excessive (hyper-) or reduced (hypo-) responses such as hormone or other protein production or other metabolic responses, administering a therapeutically effective amount of a pharmaceutically acceptable composition, to a subject in need thereof.

In certain embodiments, the cell hypersecretion is excessive hormone secretion disorder of one or more of: adrenal glands, ovaries, testes, thyroids, pituitary glands, and the like.

In certain embodiments, the cell hypersecretion is an ecotopic hormone secretion disorder.

In certain embodiments, the disorder is a cell proliferation disorder selected from one or more of: autoimmune, graft vs host (GvH) or host vs graft (HvG) diseases.

In certain embodiments, the cell proliferation disorder is cancer.

The present invention, in one specific aspect thereof, provides a novel method for treating or preventing cancer, infectious disease, autoimmune disease, asthma, allergy and transplantation rejection, by administering to a subject in need thereof a therapeutically effective amount of a pharmaceutically acceptable composition, to a subject in need thereof.

The present invention, in one specific aspect thereof, provides a novel method of inducing an adaptive immune response in a subject to a target antigen, comprising the step of: administering to a subject the complex in an amount effective to induce an adaptive immune response.

The present invention, in one specific aspect thereof, provides a novel use of autologous mature DCs formed by the method in the manufacture of a medicament for the induction of an adaptive immune response.

The present invention, in one specific aspect thereof, provides a novel method of reducing chronic inflammatory responses comprising an autologous mature IL12 producing DC produced by the method herein.

The present invention, in one specific aspect thereof, provides a novel composition for initiating an immune response comprising an autologous antigen-presenting mature dendritic cell (DC) produced by the method herein.

The present invention, in one specific aspect thereof, provides a novel method of controlling an immunodeficiency viral load of a subject, comprising the step of: administering a population of the mature DCs produced by the method described herein to the subject at a dosage and for a time sufficient to reduce the immunodeficiency viral load.

The present invention, in one specific aspect thereof, provides a novel method of inducing an immune response in a subject, comprising the step of: administering a population of the mature DCs produced by the method described herein to the subject at a dosage and for a time sufficient to induce protective immunity against subsequent infection.

The present invention, in one specific aspect thereof, provides a novel method of inducing a T cell response to an antigenic peptide in a subject in need thereof, comprising:

i) culturing precursor, or immature, dendritic cells (iDCs) from a subject in the presence of a peptide construct to provide a population of mature dendritic cells (DCs) which express a desired cytokine profile; the peptide construct having a formula P₁-x-P₂; and

ii) reintroducing the mature DCs population to the subject.

In certain embodiments, the mature DCs express interleukin 12 (IL-12).

In certain embodiments, the mature DCs are characterized by up-regulation of at least one of the following: CD11c and DC86.

In certain embodiments, a CD8 cell response is induced in the subject in need thereof.

In certain embodiments, a population of the mature DCs produces an immunomodulatory response and an increased ratio of interleukin 12 (IL-12) as compared to an iDC population not contacted with the peptide construct.

The present invention, in one specific aspect thereof, provides a novel composition for the treatment of a condition where a modulation of a Th1-mediated immune response is desired, the composition comprising at least one complex;

wherein the modulation results from a selective modulation of function of regulatory T cells and/or from a modulation of cytokine expression.

The present invention, in one specific aspect thereof, provides a novel pharmaceutical composition comprising the composition and a pharmaceutically acceptable excipient, diluent or carrier.

The present invention, in one specific aspect thereof, provides a novel composition for treating a cancerous or malignant condition comprising composition and a pharmaceutically acceptable excipient, diluent or carrier.

The present invention, in one specific aspect thereof, provides a novel method for inducing a Th1 response in a subject suitable for the treatment of a cancer or an infectious disease, the method comprising the steps of:

i) exposing isolated immature dendritic cells to a P₁-x-P₂ peptide construct to form a DC-peptide conjugate mixture; and

ii) removing free peptide construct from the mixture to form a complex, and

iii) administering the complex to a subject whereby the immune response generated in the subject is sufficient to prevent the onset or progression of cancer or to prevention infection with a pathogenic micro-organism and thereby prevent an infectious disease.

The present invention, in one specific aspect thereof, provides a novel anti-cancer vaccine complex comprising a peptide construct that binds to an immature dendritic cell.

The present invention, in one specific aspect thereof, provides a novel method of treating cancer comprising: i) obtaining an anti-cancer complex; and ii) administering the complex to a subject with cancer.

In certain embodiments, the cancer is selected from one or more of: solid cancers, epithelial cancers, mesenchymal cancers, hematological cancers, neural cancers, carcinomas, melanomas, sarcomas, neuroblastomas, leukemias, lymphomas, gliomas and myelomas.

The present invention, in one specific aspect thereof, provides a novel method for activating T cells in a subject, comprising:

i) providing precursor, or immature, dendritic cells (iDCs);

ii) contacting the iDCs with at least one P₁-x-P₂peptide construct during a time period sufficient for binding of the peptide construct to the iDCs;

iii) culturing under conditions suitable for maturation of the iDCs to form a mature dendritic cell (DC) population; and;

iv) contacting the mature DC population with T cells from the subject.

In certain embodiments, the T cells and the iDCs are autologous to each other.

The present invention, in one specific aspect thereof, provides a novel isolated population of mature dendritic cells (DCs) suitable for clinical application, preferably human mature DCs, characterized in that they: i) display a modulatory response towards T cells; and ii) are capable of producing IL-12.

The present invention, in one specific aspect thereof, provides a novel pharmaceutical composition, preferably a vaccine composition, comprising a population of mature DCs. The population can be prepared by maturation of immature DCs with a composition comprising a P₁-x-P₂peptide.

In certain embodiments, there is provided herein use of a population of mature DCs, for the manufacture of a medicament for the treatment of a condition which would benefit from immune stimulation, such as cancer or a viral infection.

The present invention, in one specific aspect thereof, provides a novel vaccine comprising the complex. In certain embodiments, the precursor, or immature, dendritic cells were originally isolated from the human subject. In certain embodiments, the peptide construct encodes a pathogen-specific antigen. Non-limiting examples of pathogen-specific antigen include, for example, an antigen from HIV, HSV, cytomegalovirus, Epstein Barr virus, human herpes virus 8, and the like.

The present invention, in one specific aspect thereof, provides a novel method of anti-tumor immunotherapy comprising: administering an effective amount of a complex, or a pharmaceutically acceptable salt thereof.

In certain embodiments, the administration is based on at least one of cancer, an elevated risk for cancer or precancerous precursors.

In certain embodiments, the administration of the complex elicits a response in at least one of tumor and cancer cells.

In certain embodiments, the response elicited is a slowing down in a growth of the tumor.

In certain embodiments, the response elicited is a reduction in a size of the tumor.

The present invention, in one specific aspect thereof, provides a novel method of immunotherapy for a subject comprising: administering an effective amount of a complex, or a pharmaceutically acceptable salt thereof.

In certain embodiments, the administration is based on an infectious disease resulting from the presence of pathogenic microbial agents

In certain embodiments, the pathogenic microbial agents are selected from the group consisting of viruses, bacteria, fungi, protozoa, multicellular parasites and aberrant proteins.

In certain embodiments, the pathogenic microbial agent is a virus.

The present invention, in one specific aspect thereof, provides a novel method of enhancing an immune response in a subject, the method comprising administering a DC-(P₁-x-P₂) complex to the subject in an amount sufficient to enhance an immune response,

wherein DC represents dendritic cells,

P₂ represents a specific antigenic peptide;

P₁ represents an immunomodulatory peptide which is a portion of an immunoprotein capable of promoting binding to a class or subclass of DC or T cells and which is capable of directing a subsequent immune response to the peptide P₂ to a Th1 or other immune response; and

x represents a covalent bond or a divalent peptide linking group, which may be cleavable or non-cleavable.

In certain embodiments, the subject has an autoimmune disease selected from the group consisting of multiple sclerosis, psoriasis, rheumatoid arthritis, and insulin-dependent diabetes.

In certain embodiments, the subject has asthma, an allergy, or a chronic inflammatory disease.

In certain embodiments, the complex is administered in a subject that has a transplantation reaction for allogeneic or xenogeneic transplants or graft-vs host disease in bone marrow transplants.

### Example 1

As described herein, the inventors examined the cytokine profiles following immunization of A/J mice with the JgD or JH vaccines or the unmodified J-ICBL. After many attempts to establish an ex vivo cell culture assay to study responses to the J-ICBL using spleen cells. The inventors herein then tested bone marrow cells, which surprisingly were shown to be responsive by cell surface marker changes, morphological differentiation and production of specific cytokines such as IL12. The inventors next analyzed the effects of J-vaccines and of the individual peptides used to make the J-L.E.A.P.S.™ vaccines on purified immature dendritic cells (iDCs) isolated from bone marrow. The inventors surprisingly discovered that J-linked vaccines activate and promote the maturation of immature DCs (iDC) and can also elicit IL-12p70 production. The cytokine profile elicited by the J-linked vaccines is different from that following DC activation through toll-like receptors (TLRs), showing that the J-L.E.A.P.S.™ vaccines activate DCs through a novel mechanism. In this embodiment, the activation required both the J and antigen specific element peptide elements covalently attached to each other.

Materials and Methods

Mice

For immunization studies, female A/J or C57BL/6 mice (Charles River, Wilmington, Mass.) were immunized, serum was obtained and pooled for analysis by cytokine protein array. Female C57BL/6 mice were used (i) to prepare bone marrow cells (Jackson Laboratories, Bar Harbor, Me.) and (ii) for generating pure DC cultures (Biological Testing Branch, Frederick Cancer Research and Development, National Cancer Institute, Frederick, Md.). All animals were treated in accordance with Institutional Animal Care and Use Committee (IACUC) approved policies and procedures.

Peptides

The JgD heteroconjugate peptide vaccine was comprised of an immune cell binding ligand, termed “J”, an amino acid 38-50 from the β-2-microglobulin having ((DLLKNGERIEKVE) [SEQ ID NO:1], conjugated to a peptide from the N-terminus of HSV-1 glycoprotein D (SLKMADPNRFRGKDLP [SEQ ID NO:2], amino acid 8-23) through a triglycine linker.

The JH heteroconjugate peptide vaccine was comprised of an immune cell binding ligand, termed “J”, an amino acid 38-50 from the β-2-microglobulin having ((DLLKNGERIEKVE) [SEQ ID NO:1], conjugated to a peptide “HGP-30 (H) peptide from the p17 HIV gag protein (YSVHQRIDVKDTKEALEKIEEEQNKSKKKA (aa 85-115)) [SEQ ID NO:3] or the through a triglycine linker.

Immunization

The peptides were dissolved in Hanks Balanced Salt Solution (HBSS) to produce a stock solution with a concentration of 2 mM adjusted to neutral pH. Each of the vaccine solutions was tested by a Limulus Amoebocyte Lysate assay as per manufacturer's instructions (Cambrex Biosciences Walkersville, Md.) and shown to be endotoxin free. The vaccine peptide was administered to mice as a 1:1 (vol) emulsion in Seppic ISA-51 (Seppic, Fairfield, N.J.). A/J or C57BL/6 female mice were immunized once with the JgD, JH, J, H, or gD peptides subcutaneously with two 50 ul injections of a 2 mM solution in the scruff of the neck and in the abdomen. The control mice were injected with HBSS in Seppic ISA-51 adjuvant.

Cytokine Protein Array Following Immunization.

Serum collected from three mice were pooled on days 3, 10, and 24 after immunizations and analyzed for 21 different cytokine and chemokine proteins using RayBio R Mouse Cytokine Antibody I array membranes as per manufacturer instructions (RayBiotech, Inc., Norcross, Ga.).

The following treatment groups were included in the analysis: 1) only adjuvant as a control group; 2) JgD in adjuvant; 3) JH in adjuvant; 4) J in adjuvant, 5) gD in adjuvant, or 6) H in adjuvant. The serum taken at each bleed was pooled such that it represents the weighted response of three animals. Presence of cytokine was detected by chemiluminescence of the membranes and the duplicate spots on film for each cytokine were analyzed by densitometry (Total Lab Array Analysis, Nonlinear Dynamics). Densitometric results were standardized for each membrane by dividing the measured value of each spot by the average values for VEGF (which should not be influenced by the treatments).

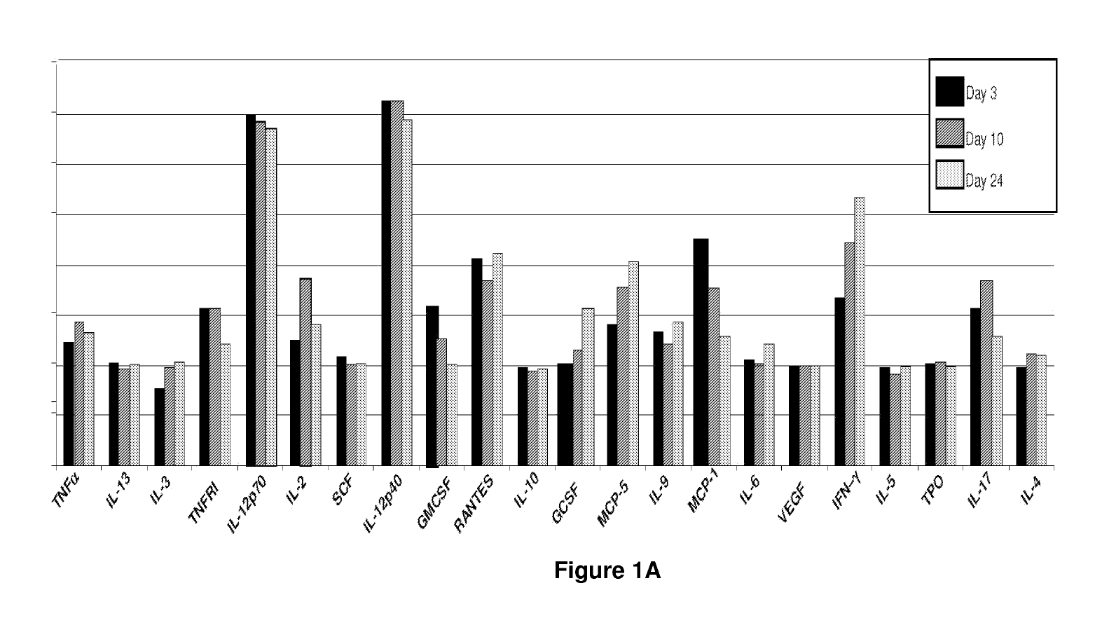

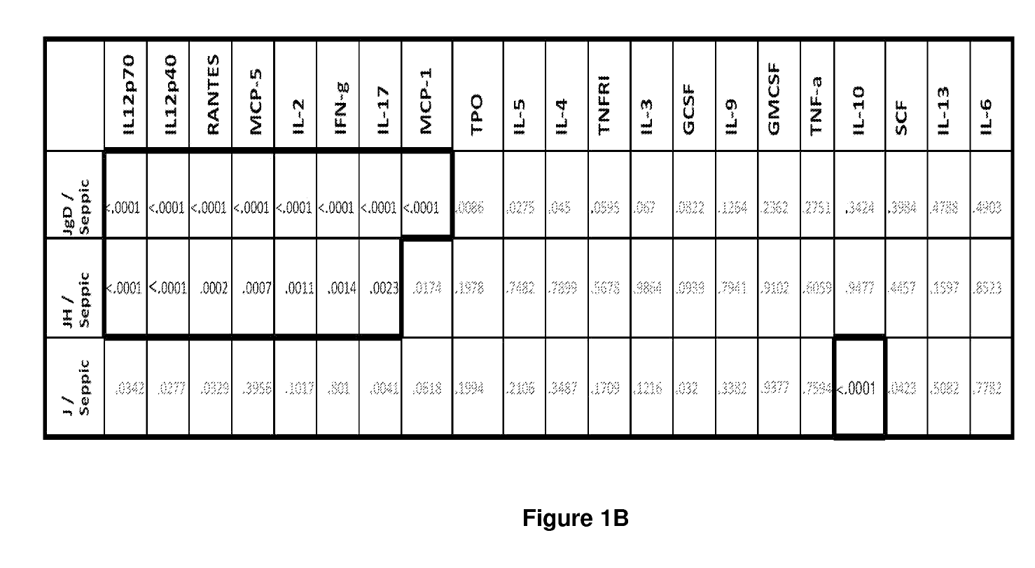

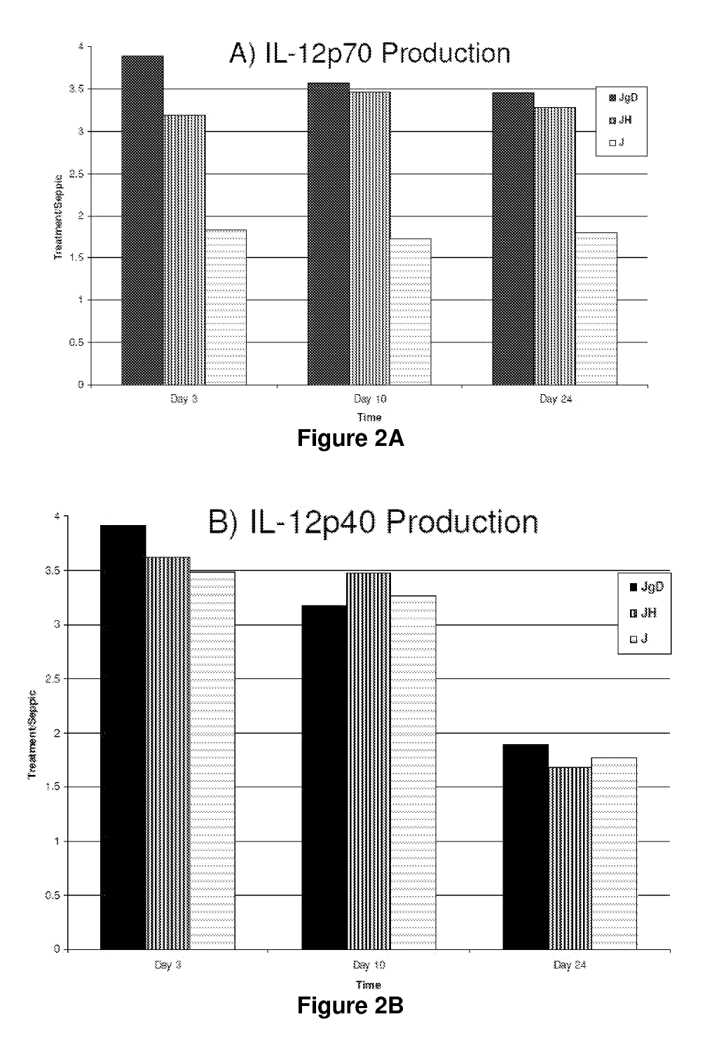

Statistical sampling was designed to maximize discovery of trends within the cytokine array results. On each membrane, 2 spots (samples) for each cytokine were measured. The replicate spots were treated as a nested source of variance rather than as replicates in the analysis to avoid pseudo-replication. Post-hoc sets for significance were performed using 2 way nested ANOVAs (SAS software system; SAS Institute, Carey, N.C.) with treatment and day as main factors. Replicate spots were not a significant source of variation. A third factor in the comparison of JgD values to adjuvant control, the strain of mouse, was not a significant source of variation for any of the 21 cytokines. A shared hypothesis (that there would be a response to a treatment) sequential Bonferroni adjustment was performed to allow for multiple comparisons. Critical alpha levels are adjusted to allow for the cumulative probability of type 1 error by this method. The data presented in FIG. 1B includes both the uncorrected P-values and indication (bold box) of statistical significance after adjustment.

Preparation of Bone Marrow (BM) Cells

Bone marrow (BM) cells were prepared. Briefly, the femurs and tibias were obtained from five C57BL/6 female mice, and the ends were removed to expose the hollow bone packed with marrow. BM cells were flushed from the bones with cold Hanks Balanced Salt Solution (HBSS) using a sterile disposable 22 g needle and pooled. Red blood cells (RBCs) were lysed using Tris-buffered ammonium chloride and resultant cells were washed 3 times in HBSS. BM cells were suspended in tissue culture medium (TCM) (RPMI 1640 with glutaminie plus 100 mg/nl PenStrep, 50 uM 2-mercaptoethanol, and 5% fetal calf serum) at approximately 5×10⁶ cells/ml and incubated for 1 hour at 37° C. in a 5% CO₂ atmosphere in plastic tissue culture flasks to remove adherent, mature macrophages. Decanted non-adherent cells were resuspended in TCM and 1.5×10⁶ BM cells in 1 ml were placed into each well of a 24-well tissue culture plate (Falcon) and either left untreated or treated with 14.5 micromoles of J, gD, JgD or JH vaccines. After incubation for 48 hrs at 37° C., cells were viewed and photographed for changes in morphology, tissue culture supernatants were removed and the cells were prepared for flow cytometric analysis.

Generation of Immature Mouse Dendritic Cells

Immature DCs were generated from the bone marrow of five normal C57BL/6 female mice. Briefly, BM cells were harvested as before and cultured at 5×10⁵/ml in 75 cm² flasks at 37° C., 10% CO₂ for 6 days in a complete media (CM) containing RPMI 1640, 10% fetal bovine serum, 2 mM glutamine, 0.1 mM nonessential amino acids, 100 units/ml sodium pyruvate, 100 mg/ml PenStrep, 0.5 mg/ml fungizone, 50 ug/ml gentamicin, 50 um 2-mercaptoethanol, supplemented with 10 ng/ml of human IL-6 (Peprotech, Rocky Hill, N.J.) and 10 ng/ml human Flt-3 (gift of Amgen, Thousand Oaks, Calif.). On day 6, the cells were washed twice in Dulbecco's PBS, 4 x 10₆ cells/well were transferred to a 24-well cluster plate and cultured in CM supplemented with 10 ng/ml of human GM-CSF (gift of Immunex, Seattle, Wash.), and incubated for 24 hrs. Cells were then analyzed by flow cytometry for expression of CD11c, CD80, CD86, MHC II, CD34, and OX40L, confirming the purity or the iDC population.

Immature DCs were either untreated or treated with 3.625, 7.25, or 14.5 micromoles of JgD peptide and maintained in CM without GM-CSF. After 48 h incubation, spent medium was removed and immediately tested for the presence of IL-12p70 by direct ELISA.

Co-Cultivation of Immunogen-Treated Bone Marrow Cells with Spleen Cells

Bone marrow cells (2×10⁶ in one ml) pooled from 3 female C57B1/6 mice were prepared and untreated or treated with JgD or JH (14.5 uM) as described above and incubated for 48 hours. At this time, spleen cells (2×10⁷), prepared and pooled from 3 mice, were either: 1) untreated; 2) treated with JgD peptide or JH peptide; 3) added to wells containing the untreated BM cells, JgD treated BM cells (JgD-BM) or JH treated BM cells (JH-BM); or 4) added to wells containing untreated BM, JgD-BM or JH-BM cells that had been washed twice (to remove unbound immunogen and extracellular cytokines) and then resuspended in 4 ml of medium. Aliquots of medium were obtained after an additional 48 hours and evaluated for cytokine production by cytokine protein array. Densitometric results for duplicate cytokine or chemokine spots from two independent trials were obtained and normalized to the summation of values for each array. The average of these duplicate results are presented as a fold increase or decrease compared to the untreated control.

Co-Cultivation of Immunogen-Treated Bone Marrow with Spleen Cells from JgD Immunized Mice.

C57BL/6 mice were immunized subcutaneously with JgD in Seppic ISA-51 and received a booster one month later. Bone marrow cells from 3 mice were pooled and 2×10⁶ cells in one ml were treated with JgD or JH (14.5 uM), as described above, and incubated for 48 h. The bone marrow cells were then washed twice to remove unbound immunogen and resuspended with JgD immunized spleen cells (JgD-Sp). Spleen cells (2×10⁷), prepared and pooled from 3 JgD immunized mice were either: 1) untreated; 2) added to JH-BM; or 3) added to JgD-BM. Aliquots of medium were obtained after a 48 h incubation period and analyzed for IFN-γ production, by ELISA, or for cytokine and chemokine production by cytokine protein arrays.

ELISA

Sera (100 microliters) collected on days 3, 10, and 24 after immunization with J, gD, JgD, or JH J-LEAPS vaccine were analyzed for IL-12p70 by a direct ELISA (Sigma, St. Louis, Mo.). Spent medium (100 microliters) was also obtained from cultures of mouse DCs at 48 h after treatment with JgD L.E.A.P.S.™ heteroconjugate and tested for IL-12p70. The ELISA was repeated and each ELISA sample was run in triplicate.

Flow Cytometry Analysis and Cell Sorting

For analysis of CD11c and CD86 expression, untreated and peptide treated BM cells, prepared and treated as described above, were labeled with PE-anti-Cdl lc or PE-anti-CD86 (Beckman Coulter Fullerton, Calif.). At least 10⁶ cells were analyzed (Altra FACS, Beckman Coulter) using forward and side scatter parameters to limit (gating) the immunofluorescence analysis to cells of the size and granularity of monocytes and dendritic cells.

CD3+ cells were removed from BM cells using the fluorescence activated cell sorter and then untreated or treated with JgD or JH. Flow cytometric analysis of the sorted population confirmed the removal of CD3 positive cells. The CD3− BM cells were labeled with FITC-anti-CD8 (Beckman Coulter (clone 53-6.7)), fixed with paraformaldehyde, permeabilized with saponin (Intraprep, Immunotech), labeled with PE-anti-IL-12p70 (Beckman Coulter) and then post fixed with paraformaldehyde prior to immunofluorescence analysis.

Treatment of mouse bone marrow cells with either JgD or JH generated DCs which produce IL-12 and also induce small quantities of interferon γ from T cells from MHC compatible spleen cells. This action would steer the immune response towards a Th1 response which includes cell mediated and antibody responses.

Mouse bone marrow cells treated with JgD enhanced the interferon γ response of T cells obtained from a mouse previously immunized with JgD as an indication of an antigen specific booster response. Mouse bone marrow cells treated with JH did not elicit this booster response. These results prove that the JgD treated bone marrow cells are sufficient to deliver an antigen specific immune response. It establishes that the LEAPS peptide stays on the surface of the DC for long periods and can interact with T cells to elicit the response.

Results for Example 1

Cytokine Production Due to J-LEAPS Immunizations

A survey of the cytokine response to J-LEAPS vaccines and its time course were obtained by cytokine protein array analysis following immunization with HBSS-Seppic ISA51 or equimolar amounts of the J-ICBL, or the JgD, gD, H, JH peptides emulsified in Seppic ISA51 adjuvant. The protein array is a sensitive semiquantitative method for simultaneous evaluation of serum levels of multiple cytokines useful for comparison of responses. The immunization schedule, amounts of vaccine and adjuvant were the same as used in experiments that demonstrated protection from lethal herpes simplex virus challenge induced by immunization with JgD. Each of the vaccine solutions was tested by a limulus amoebocyte lysate assay and shown to be endotoxin free (data not shown). Cytokine array results for serum from mice immunized with HBSS in Seppic ISA51 adjuvant were unremarkable. Similarly, no cytokine response was detected at 3, 10 or 24 days following immunization with the H or gD peptides in Seppic ISA-51 (data not shown).

FIGS. 1A and 1B show the ratio of mean values for serum cytokine production for C57BL/6 and A/J female mice following immunization with JgD to values obtained for mice immunized with adjuvant alone. The time course and trends for the values representing serum levels of cytokines generated by immunization of C57BL/6 and A/J mice with JgD were not significantly different and are presented as an average.

The Table in FIG. 1B identifies the cytokines or chemokines produced in significantly different amounts (surrounded by bold box) by A/J mice immunized with JgD, JH or J compared to adjuvant treated mice as per sequential Bonferroni analysis of the uncorrected ANOVA values for multiple contrasts including treatment type and day post treatment.

By the third day after immunization, elevated levels of cytokines and chemokines associated with DC1 innate responses were observed. Amounts of IL-12p40 and IL-12p70 (p40+p35) increased to levels approximately 3 to 4 times higher than for mice immunized with adjuvant alone. There was also a two-fold increase in GM-CSF, MCP1 and RANTES. Interestingly, cytokine levels of TNFα and IL1 were not increased by immunization with JgD. Small increases in IL-17 and IFN-γ were present on day 3 but these values were not significant until day 10. The levels of IL-12p40 and IL-12p70 remained elevated on the 10th and 24th days after immunization with JgD accompanied by significantly increased levels of IFN-γ. Levels of MCP1 decreased while levels of MCP5 increased over this time period. IL-17 levels were also significantly elevated on the tenth day but receded by day 24. Early production of IL-12p40 and IL-12p70 with subsequent production of IFN-γ in response to immunization with JgD is consistent with generation of DC1s which activate T cells.

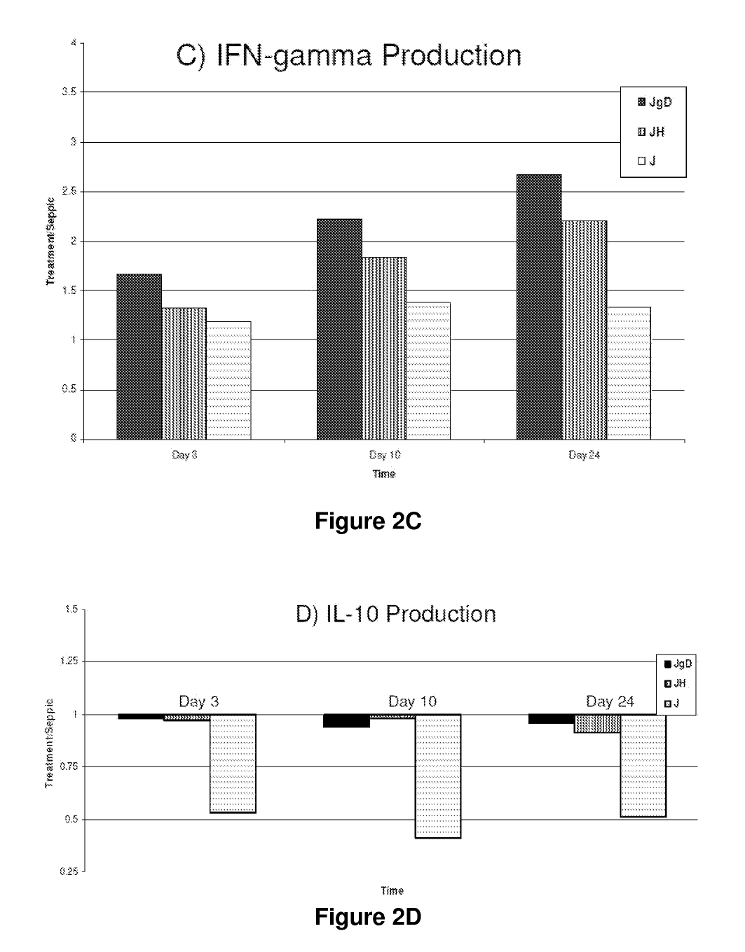

The overall response to immunization of A/J mice with JH was not statistically different from the response to JgD (p-value=0.849) but the values for JgD and JH were significantly different from the adjuvant control treated mice (p<0.001) and from J treated mice (p<0.05). As for JgD, levels of RANTES, MCP-5, IFNg and IL17 were elevated over the 24 day course of immunization but TNF-α and IL-6 levels were not affected by immunization with the JH vaccine. Immunization of A/J mice with the J-ICBL caused different results than JgD or JH (see FIGS. 2A-2D).

There was a small but not significant increase in IL-12p40 and IL-12p70. Interestingly, IL-10 levels were significantly decreased to only a half or to a third as much as the adjuvant control. No other remarkable effect was observed following immunization with the J-ICBL.

The same sera that were analyzed by cytokine protein arrays (see FIGS. 1A-1B) were also quantitated by a direct ELISA for IL-12p70. Whereas only trace or undetectable amounts of IL-12p70 were present in sera from mice immunized with J, gD, H, or HBSS-Seppic ISA51, the sera obtained from JgD immunized mice contained 378 pg/ml, 353 pg/ml and 372 pg/ml of IL-12p70 on days 3, 10 and 24, whereas sera obtained from JH immunized mice contained 334 pg/ml, 376 pg/ml and 386 pg/ml of IL-12p70 on days 3, 10 and 24. These results are consistent with the elevated levels of IL-12p70 detected by the cytokine protein array. The similarity in IL12p70 response for JgD and JH suggests an antigen independent action of immune cells.

Ex Vivo Analysis of Bone Marrow Cell Response to J-LEAPS Immunogen

An ex-vivo assay system utilizing BM cells was developed to further study the response to the J-LEAPS immunogens. Bone marrow is a good source of stem cells for naïve myeloid DCs with few or no T cells.

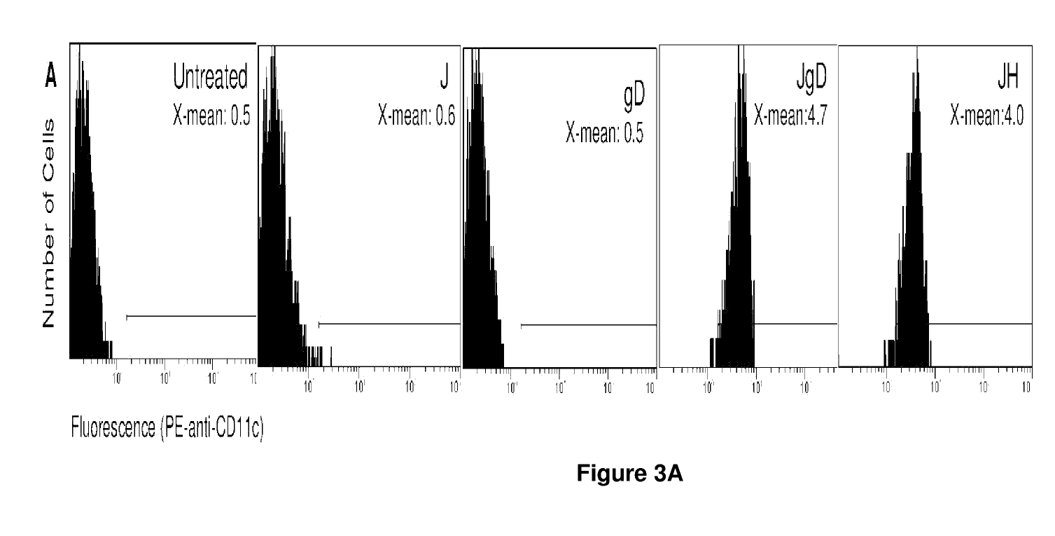

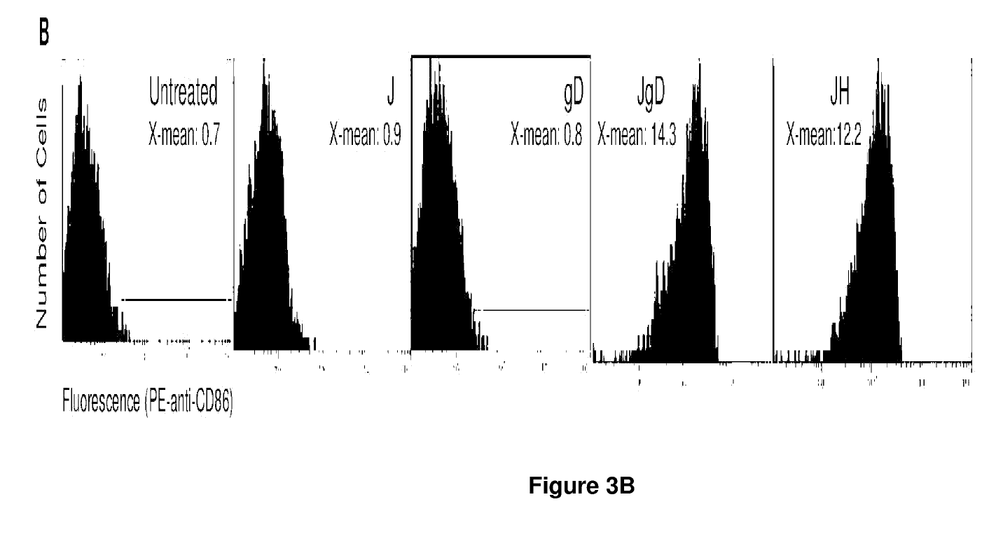

The monocyte population of BM cells, as defined by light scatter parameters, was analyzed on the second day after treatment with J, gD, JgD or JH. Representative flow cytometric results are presented in FIGS. 3A-3B.

The untreated monocyte population contained very few CD11c or CD86 positive cells whereas the JgD-BM and JH-BM expressed CD11c (FIG. 3A) and CD86 (FIG. 3B). CD11c is a type I transmembrane protein found on most human and mouse dendritic cells and CD86 is a cell marker for mature DCs capable of signaling and activating T cells.

Treatment with gD or the J-ICBL caused no discernable change in CD11c or CD86 expression. Similarly, there was no significant increase in IL-12p70 expressing cells following J-ICBL treatment. Protein array analysis of spent medium from J-ICBL treated BM cells failed to detect a cytokine response (data not shown).

In a separate experiment, CD3 expressing T cells were removed by FACS and the remaining bone marrow cells were incubated with JgD or JH and analyzed for surface CD8 and intracellular IL-12p70 expression (FIGS. 3C-3D).

After 48 hours, only 12% of the control cells expressed low levels of CD8 and less than 3% of these cells were positive for IL-12p70. The JgD-BM CD3⁻ population contained 73% CD8 positive, IL-12p70 producing cells and the JH-BM CD3− population contained 72% CD8 positive, IL-12p70 producing cells. Treatment with JgD or JH appears to increase the number of CD8 expressing cells or the expression of CD8 and promotes IL-12p70 production in this population of bone marrow cells. Production of IL-12p70 in CD3(−)/CD8(+) expressing cells proves that the JgD and JH responsive cells are of myeloid origin and not T cells.

Effects of JgD on an Isolated Dendritic Cell Population

Production of IL-12p40 and IL-12p70 following immunization of mice with either the JgD or JH immunogens and the generation of mature DCs upon treatment of BM suggests that these J-LEAPS vaccine peptides are sufficient to directly activate immature DCs (iDCs) and generate IL-12 producing DC1s. To demonstrate that iDC are the target for the J-LEAPS peptides, the effect of JgD was tested on iDCs prepared from BM.

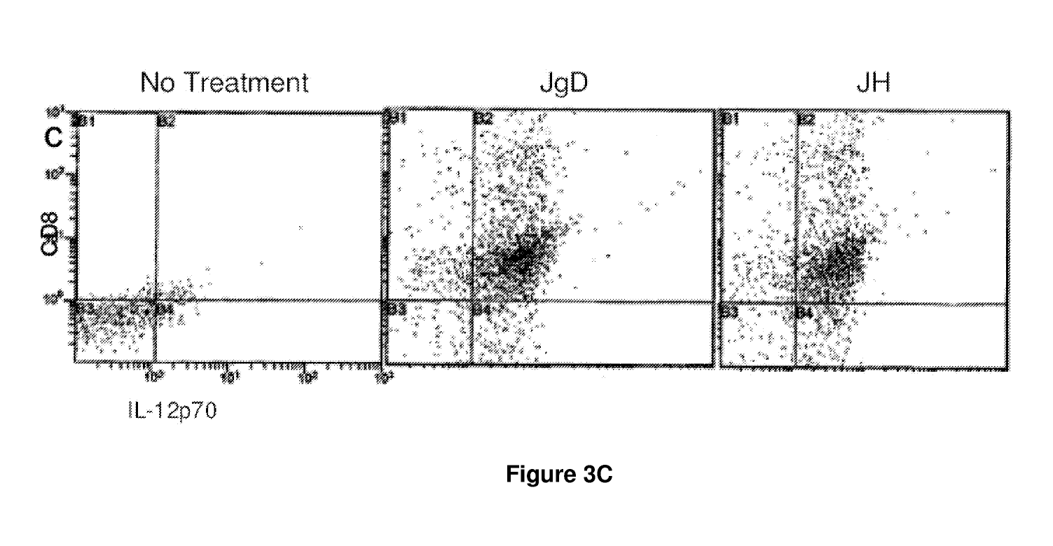

Morphological changes were observed within 48 hrs of addition of JgD to the immature dendritic cell culture (FIG. 4A). The cells clustered together and spindling dendrites formed, both of which are characteristics of maturing dendritic cells.

Treatment with JgD also promoted a measureable, concentration dependent increase in IL-12p70 production (FIG. 4B).

Cells (1.5×10⁶) treated with 3.625 micromoles of JgD produced 8.0 ng of IL-12p70, whereas cells treated with 7.25 micromoles of JgD produced 9.50 ng and treatment with 14.5 micromoles of JgD yielded 12.5 ng of IL-12p70. The change in morphology and production of IL-12p70 indicate that treatment with the JgD immunogen is sufficient to activate the development of iDCs into DC1s.

JgD- or JH-Activated BM-Derived DCs Activate Splenic T Cells to Produce Interferon γ.

JgD or JH treated BM cells were incubated with spleen cells, as a source of T cells, and evaluated for IFN-γ production to test whether the J-LEAPS immunogen treated bone marrow cells were converted into DC1 cells capable of activating T cells. Spleen cells were either: 1) untreated; 2) treated with JgD peptide or JH peptide; 3) added to wells containing the untreated BM cells, JgD treated BM cells (JgD-BM) or JH treated BM cells (JH-BM); or 4) added to wells containing untreated BM, JgD-BM or JH-BM cells that had been washed twice and resuspended in fresh medium.

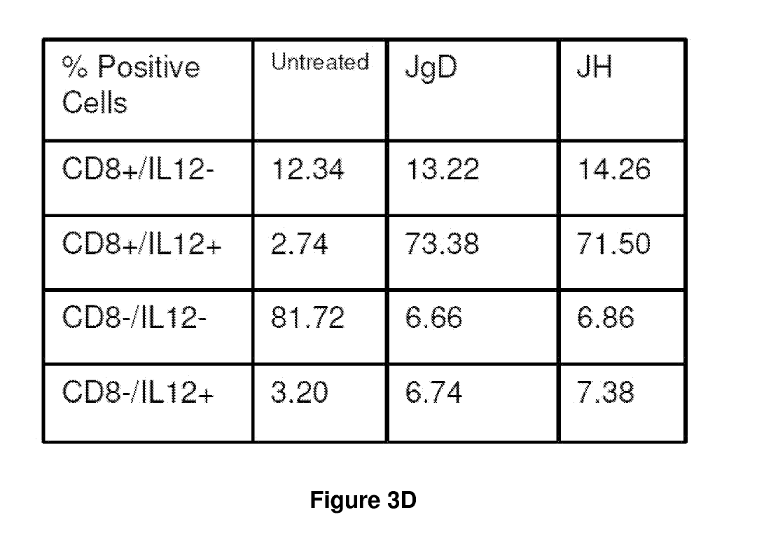

As seen in FIG. 5, elevated levels of IL-12, MCP 1, MCP 5, RANTES, IL-2, and IFN-γ were present in spent media after spleen cells had been incubated with JgD-BM or JH-BM for 48 h compared to untreated spleen cells. The presence of IL-2 and IFN-γ are characteristic of a Th1 response. Removing unbound immunogen and any previously produced cytokines by washing the treated bone marrow cells prior to addition of spleen cells did not significantly reduce the amount of IFN-γ production. IFN-γ was not produced by spleen cells when untreated or following treatment with the cell-free JgD or JH peptides.

Consistent with previous experiments, there was no detectable IL-6 or TNF-α in the media following T cell incubation with JgD-BM or JH-BM. Interestingly, media from the spleen cells incubated with untreated bone marrow cells had elevated but low levels of IL-4, IL-5, IL-10, and IL-13 (data not shown), suggestive of a Th2 type of response, while no detectable amounts of these cytokines were present in the samples from spleen cells mixed with the JgD-BM or JH-BM.

These results demonstrate that treatment with JH or JgD is sufficient to convert precursor cells into DC1 cells and the treated DC1 cells are capable and sufficient to activate T cells to produce the Th1 defining cytokines, IFN-γ and IL-2.

In order to determine whether the JgD-BM cells were also capable of antigen presentation to T cells to promote a specific vaccine-like immune response, spleen cells obtained from mice (n=3) immunized with JgD, as described for FIG. 1, were incubated with JgD-BM or JH-BM (prepared as described for FIG. 5).

Spent medium was obtained after an additional 48 h (96 h after JgD or JH treatment of BM) and cytokine levels were analyzed by protein array and IFN-γ levels were also analyzed by ELISA (Table 1).

TABLE 1
A. Cytokine response of co-cultures of
JgD immunized splenocytes with JgD or JH treated bone marrow cells.

| | | |
|---|---|---|
| | JH-BM + JgD-Sp/ | JgD-BM + JgD-Sp/ |
| CYTOKINES | Untreated | Untreated |

Protein Array^{1,2}

| | | |
|---|---|---|
| TNF-α | 0.00 | 0.00 |
| IL-12p70 | 4.48 | 7.15 |
| IL-2 | 4.33 | 7.27 |
| IL-12p40 | 4.53 | 7.21 |
| RANTES | 4.43 | 7.05 |
| MCP-5 | 4.27 | 6.85 |
| MCP-1 | 4.17 | 6.77 |
| IL-6 | 0.00 | 0.00 |
| IL-17 | 4.06 | 6.62 |
| IFN-γ | 4.27 | 11.83 |

B. Cytokine response of co-cultures of JgD immunized
splenocytes with JgD or JH treated bone marrow cells.
ELISA³

| | | |
|---|---|---|
| IFN-γ ELISA³ | undetectable | 495 pg/ml |
| | (<150 pg/ml) | |

¹Average of (2 independent experiments) selected values from protein array of samples obtained at 48 h after culture of splenocytes from immunized (JgD-Sp) without or with either JgD treated (for 48 h prior to co-incubation) bone marrow cells (JgD-BM) or JH treated bone marrow cells (JH-BM). Cytokine array analysis was performed as described in FIG. 5.
²All p values were <1.8 × 10⁻⁴ per ANOVA
³ELISA result for JgD immunized spleen cells co-cultured with untreated bone marrow cells was zero.

Cytokine responses of the JgD immunized spleen cells to JH-BM were similar to the previous experiment (see FIG. 5) and similar to responses to JgD-BM except for the IFN-γ response. Increases in IFN-γ production in response to JH-BM were noted by protein array but were undetectable by ELISA. Importantly, IFN-γ production was elevated for JgD-BM and also detectable by ELISA. This is consistent with the occurrence of an antigen specific booster response of the JgD-splenic T cells mediated by JgD-BM.

### Discussion of Example 1

The J-ICBL, in the LEAPS approach to vaccine development, converts epitope containing peptides that are too small to elicit a response into immunogens. For an immunologically naïve mouse, these heteroconjugate peptides have generated immune activities that are consistent with Th1 responses including immunoglobulin subtype production, antigen specific DTH, and protection from lethal HSV challenge.

Analysis of the cytokine profile produced in response to immunization of mice with the JgD and the JH immunogens is consistent with DC1 cells promoting a Th1 response. Similar responses were observed in two different mouse strains and for two different J-vaccines.

A DC1 produces IL-12p70 and presents antigen to T cells to promote the development of a Th1 response and consistent with the development of DC1 cells, IL-12p70 was present within the first 3 days of immunization of mice with the JgD or JH vaccines in Seppic ISA51. There was a concomitant increase in IL-12p40.

The response progressed to include increased production of IL-17 and IFN-γ ten days after treatment with either JgD or JH, but not with the J-ICBL. Lack of an epitope containing portion attached to the J-ICBL appears to preclude the production of the T cell-associated cytokine response. The IL-17 response was transient and decreased by day 24. IL17 production may result from early IL-23 production (not assayed). IL-12p70 and IL-23 are heterodimeric proteins and both utilize the same p40 subunit but with different p35 or p19 subunits, respectively. As levels of IL-12p70 increased, it may have inhibited the production of IL17.

The cytokine response to JgD or JH treatments of the mice differed from that induced by better known activators (TLRs) of DCs, such as LPS or Lipid A (MPL), CPG-ODN, in which acute phase cytokines are usually produced when IL-12p70 is produced. In contrast, the amounts of IL-12p70 produced in response to JgD or JH were modest and were not accompanied by increases in IL-6 nor TNF-α production. These differences distinguish the nature of the response to J-LEAPS immunogens from those activated by TLR activating ligands.

The JgD, JH, and J peptides were administered to the mice in an emulsion with Seppic ISA51 to mimic conditions used in earlier vaccine protection studies. By itself, the Seppic ISA 51 adjuvant did not activate a relevant cytokine response and the adjuvant was not required for generation of IL-12p70 producing DC1 cells from BM cells by JgD or JH treatment, ex vivo. The longevity of the IL-12p70 and IFN-γ responses in the immunized mice suggest that the Seppic ISA51 adjuvant establishes a slow release reservoir for the vaccine. A depot effect would sustain the response and may explain its role in the immune protection from lethal HSV challenge elicited by immunization with JgD.

The J-ICBL has biological activity but is not antigen specific and is insufficient to induce the response associated with JgD or JH. Evidence of the biological activity of J-ICBL was the significant reduction in serum levels of IL-10 to less than half the amount of mice treated with adjuvant alone. The J-ICBL cannot elicit anti-J antibody production nor did it elicit detectable cytokine production from BM cells and cannot promote differentiation of BM into DCs.

J may have an effect on cells other than those present in BM to reduce serum IL-10 levels. IL-10 is an immunosuppressive cytokine and one that promotes Th2 responses. Reduction of IL-10 levels would also promote an environment that is more conducive to activation of Th1 immunity.

Covalent attachment of the J-ICBL to the epitope bearing peptide is necessary for induction of Th1-like immune responses. In previous studies of LEAPS-based vaccines, neither the epitope containing peptide, the J-ICBL by itself, nor an unconjugated mixture of the peptides was able to produce specific antibody. Neither the gD nor H peptides could elicit cytokine responses in mice or in cell culture despite use of equimolar amounts to the corresponding JgD and JH heteroconjugate vaccines.

While not wishing to be bound by theory, the inventors herein believe that the covalent linkage within the LEAPS heteroconjugate immunogen may be necessary to promote crosslinking of an epitope-binding MHC molecule and a J-binding receptor on the cell surface to activate a maturation cascade to produce a DC1.

Immunization with peptides attached to other ICBLs, such as “L”, a peptide from ICAM, “G”, a peptide from the β chain of MHC II, or ‘F’, from IL-1β, elicit no response, Th2, or mixed responses, respectively that are very different from those elicited by heteroconjugates with the J-ICBL.

It was surprising that the J-LEAPS immunogens were sufficient to induce maturation of bone marrow cells into CD8 expressing IL-12p70 producing cells with the morphology and increased expression of CD86 and CD11c of DC1s. Bone marrow cells include precursors of monocytes which are also precursors of DCs. Induction of maturation of the BM cells into DCs by JgD or JH occurred without the need for an adjuvant, cofactor, or T cells. The inventors herein have now also shown, with human monocytes, that JgD and JH can also promote the maturation of human precursors into DC1s. The JgD treatment of mouse BM appeared to also increase the expression of CD8 on the DC population. By stimulating CD8 expressing iDCs to mature, the J-LEAPS vaccines are activating a type of murine DC that is associated with cross priming of antigen to CD8 T cells and these DCs also produce IL-12p70.

Ultimately, the J-immunogens must be able to generate a DC1 cell that can present antigen and promote production of Th1 cytokines from T cells. As demonstrated, the JgD-BM or JH-BM were sufficient to promote production of IFN-γ and IL-2, the prototypic Th1 cytokines, from splenic T cells whereas untreated T cells produced low levels of Th2-related cytokines, such as IL-4, IL-10 and IL-13. Interestingly, the lack of response of spleen cells and splenic T cells to JgD or JH reiterates the relevance of DC precursors, not mature DCs or T cells, as the initial target during immunization with J-LEAPS immunogens.

Ultimately, the JgD and JH activated DC1s appear to be capable of producing sufficient IL12p70 to steer the response of T cells and activate a generic low level of IFN-γ production.

Proof that JgD can act as an immunogen was demonstrated by the antigen specific boost in IFN-γ response, which followed incubation of JgD-BMs but not JH-BMs, by T cells from JgD immunized mice. Induction of the antigen specific booster response also indicates that the JgD is retained on the cell surface of the DC1s to interact with the TCR and was sufficient to induce the antigen specific response from T cells of the JgD immunized mice.

The ex-vivo studies with BM cells demonstrate that J-LEAPS immunogens act as both an adjuvant, to stimulate differentiation of precursors into DC1s, and as an antigen, capable of interacting with MHC molecules and being presented to antigen specific T cells. By activating the maturation of DC1s and their production of IL-12p70, the J-LEAPS vaccines define the direction of the immune response. The lack of acute phase cytokine production denotes a unique pathway of DC activation, one which should allow immunomodulation with potentially less immunopathology. The ability of the J-LEAPS heteroconjugate peptides to activate DCs that elicit an antigen specific Th1 response without the need for an additional TLR ligand provides a clean approach to designing a vaccine capable of eliciting appropriate protective responses.

### Example 2

The inventors treated human GMCSF (G-monocytes), GMCSF plus IL4 pulsed (G4-monocytes), or untreated monocytes with immunogens developed by the LEAPS technology. The ligand antigen epitope presentation system (L.E.A.P.S.) converts small peptides into immunogens by chemical conjugation to an immune cell binding ligand (ICBL) such as J ((DLLKNGERIEKVE) [SEQ ID NO:1], amino acid 38-50 from the β-2-microglobulin). The JgD and JH heteroconjugate peptide immunogens consist of a peptide from the N-terminus of HSV-1 glycoprotein D (SLKMADPNRFRGKDLP [SEQ ID NO:2], amino acid 8-23) or the HGP-30 (H) peptide from the p17 HIV gag protein (YSVHQRIDVKDTKEALEKIEEEQNKSKKKA [SEQ ID NO:3] (aa 85-115)) conjugated to the J-ICBL through a triglycine linker. We show that these LEAPS immunogens can promote the maturation of monocytes into IL12-producing DCs.

Materials and Methods

Human Monocyte Preparation and Purification

Monocytes (>95% pure) were collected by leukapheresis (Baxter CS 3000) (Apheresis unit, Cleveland Clinic Foundation), followed by elutriation (Beckman Elutriator), washed and frozen After thawing, cells were plated at 3×10⁶ cells/ml in monocyte-macrophage serum free medium (Life Technologies, Gaithersburg, Md.) with or without 50 ng/ml recombinant human GMCSF (Immunex, Seattle, Wash.) (GM-monocytes) or GMCSF+ 500 U/ml recombinant human IL4 (Schering-Plough, Bloomfield, N.J.) (GM-4 monocytes) for 24 h at 37° C. After 24 h, the cells were treated with 14.5 μmol of JgD, JH, J, gD, or H peptides or HBSS.

J-LEAPS™ Immunogens

Peptide immunogens synthesized by UCB (Atlanta, Ga.) and supplied by Cel-Sci (Vienna, Va.) were dissolved in Hanks Balanced Salt Solution (HBSS) to produce a stock solution with a concentration of 2 mM adjusted to neutral pH. Each of the vaccine solutions (100 μl) was tested by a Limulus Amoebocyte Lysate assay as per manufacturer's instructions (Cambrex Biosciences Walkersville, Md.) and shown to be endotoxin free.

Cytokine Arrays

Medium from peptide treated and untreated cells were obtained after 3 days and assayed for the presence of 42 different cytokine and chemokine proteins using RayBio® Human Cytokine Antibody Array 3 membranes (RayBiotech, Inc., Norcross, Ga.). Cytokines were detected by chemiluminescence, and the results captured on X-ray film were analyzed by densitometry (Total Lab Array Analysis, Nonlinear Dynamics).

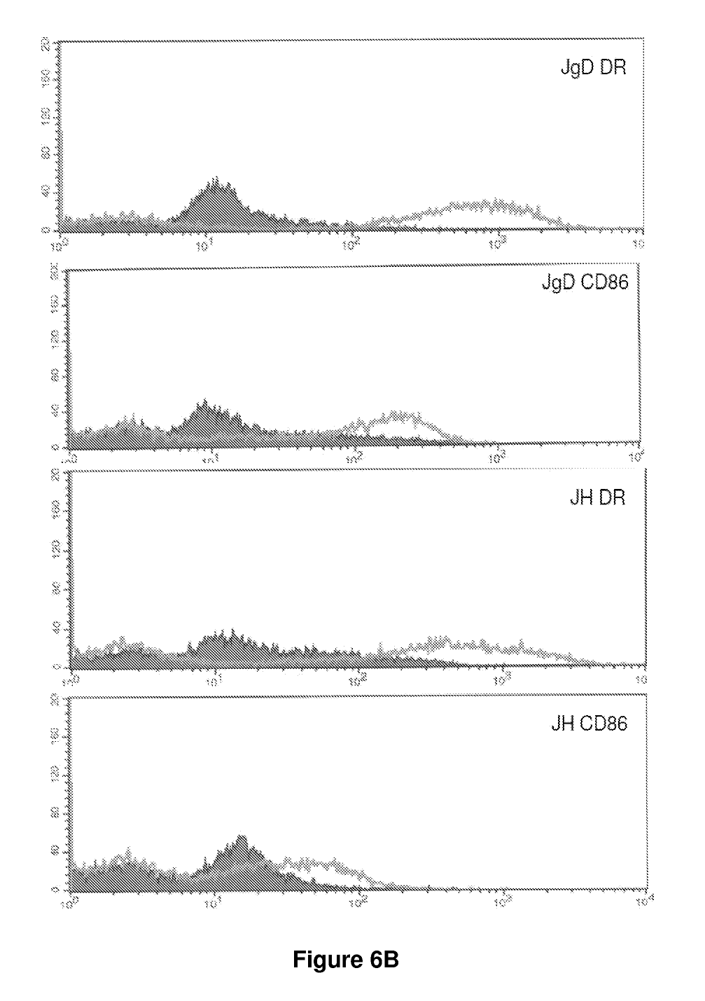

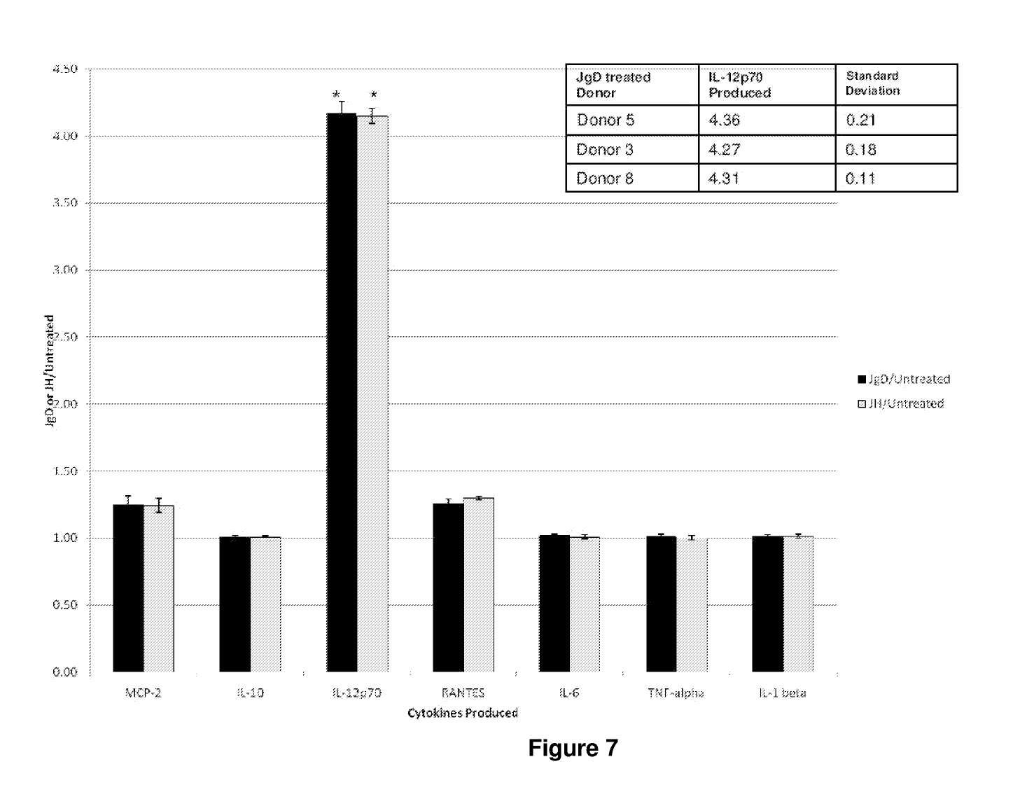

In FIG. 7 and FIG. 8 array results were quantitated by densitometry, and normalized to the summation values for each array to allow for comparative analysis of JgD or JH treated to untreated dendritic cell array results. These values were then compared to the values obtained for untreated supernatants and results presented as a fold change. Statistical analysis for significant differences for each comparison was performed by equating p-values via ANOVA analysis. In Table 2, densitometric results for each cytokine were divided by the results for EGF (which should not be affected by treatment) to allow comparison of results between array samples.

TABLE 2

| Cytokine | Monocyte | GMCSF | GMCSF + IL4 |
|---|---|---|---|
| IL12p70 | 3.09^{a} | 3.05 | 3.58 |
| MCP-1^{b} | 2.54 | 2.45 | 0.80 |
| MCP-2 | 2.45 | 2.40 | 1.84 |
| RANTES | 1.61 | 1.51 | 1.57 |
| PDGF-BB | 1.38 | 1.44 | 1.25 |
| MIP-1 delta | 1.15 | 1.10 | 1.64 |
| ENA-78 | 0.77 | 0.76 | 0.74 |
| MCSF | 0.68 | 0.60 | 1.49 |
| MDC | 0.68 | 0.67 | 1.89 |
| MIG | 0.37 | 0.37 | 0.39 |
| Angiogenin | 0.60 | 0.59 | 1.00 |
| Oncostatin | 0.52 | 0.50 | 1.17 |
| TARC | 0.22 | 0.24 | 0.47 |
| VEGF | 0.15 | 0.21 | 1.49 |
| GCSF | 0.00 | 0.00 | 1.22 |
| IL1α | 0.00 | 0.00 | 0.36 |
| IL10 | 0.00 | 0.00 | 0.34 |
| TNFα | 0.00 | 0.00 | 0.27 |
| IL1β | 0.00 | 0.00 | 0.23 |

^{a}Densitometric values were normalized to EGF for standardization between cytokine protein arrays.
^{b}MCP-1 and -2, monocyte chemoattractant proteins; RANTES, regulated upon activation normal T cell express sequence; PDGF-BB, platelet derived growth factor; MIP-1 delta, macrophage inflammatory protein-1 delta; ENA-78, epithelial neutrophil activating peptide 78; MCSF, macrophage colony-stimulating factor; MDC, macrophage derived chemokine; MIG, monokine-induced by interferon γ; TARC, thymus and activation regulated chemokine; VEGF, vascular endothelial growth factor; GCSF, granulocyte colony-stimulating factor; EGF, epidermal growth factor; GMCSF, granulocyte macrophage colony-stimulating factor.

Flow Cytometry Analysis

Untreated and immunogen treated monocytes were labeled with PE-anti-DR or PE-anti-CD86. At least 5×10⁵ cells were analyzed by flow cytometry (FACS Calibur; Cell Quest Pro software) (BD Biosciences San Jose, Calif.).

Allogeneic Mixed Leukocyte Cultures

Monocytes harvested 24 h after treatment with JgD or HBSS were co-cultured with CD4 T cells, obtained as a byproduct of elutriation and purified by negative selection (T cell isolation columns; R&D, Minneapolis, Minn.) (1×10⁶ cells), at a monocyte: T cell ratio of 1:10 for 6 days at 37° C. in RPMI 1640 medium supplemented with 5% human AB serum (Cambrex, East Rutherford, N.J.). Culture supernatants were collected and assayed via RayBio® Human Cytokine Antibody Array 3 for cytokine production.

### Results for Example 2

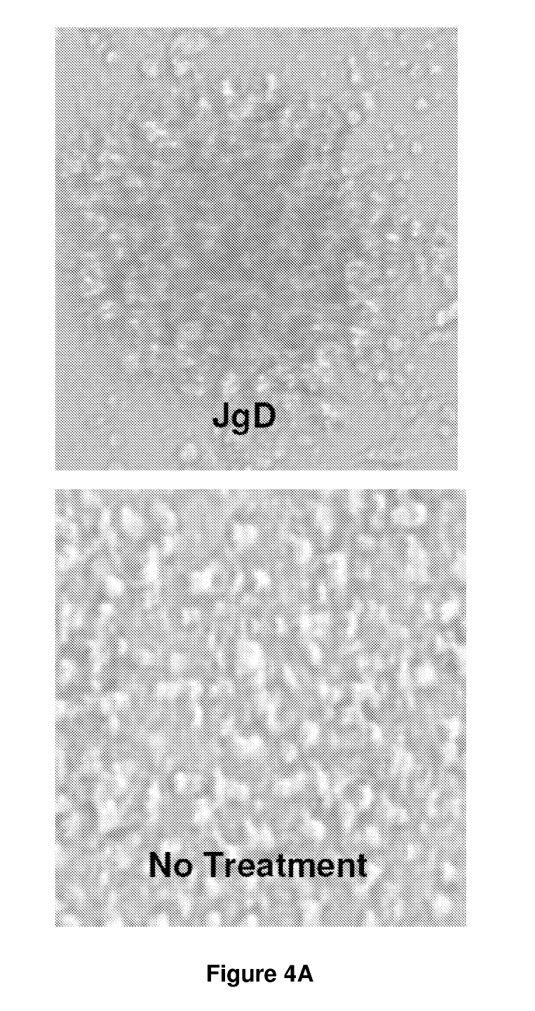

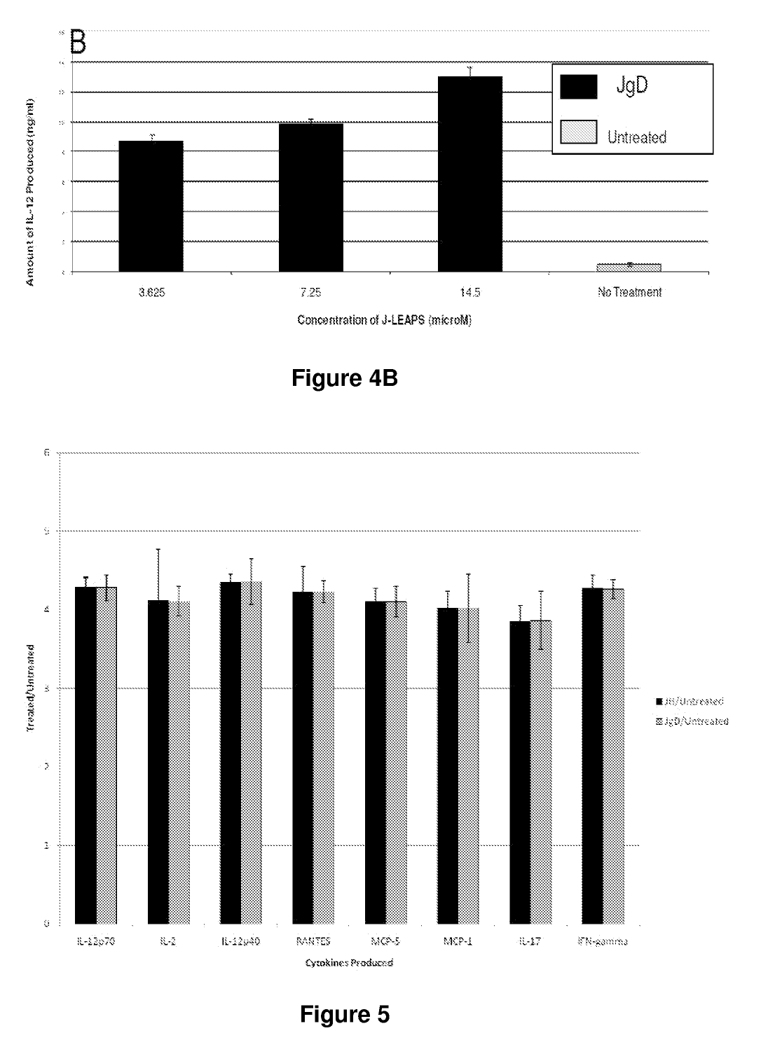

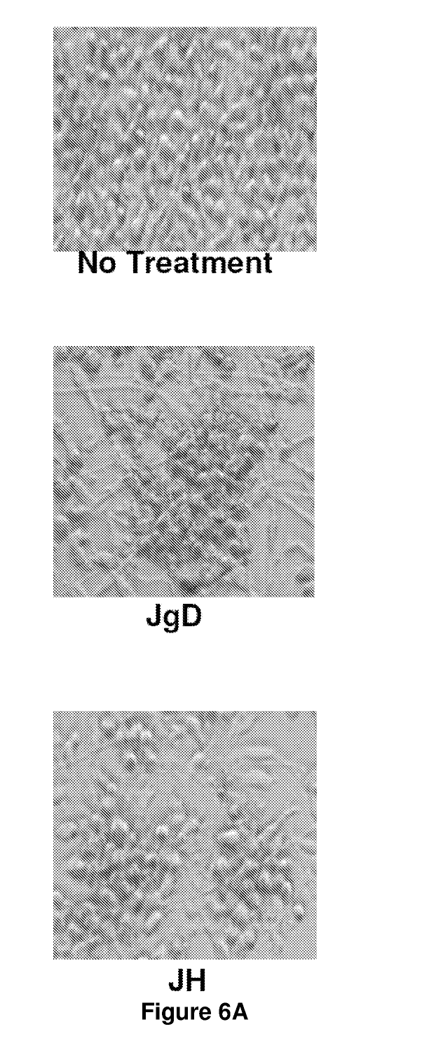

The inventors herein determined whether JgD and JH will promote the maturation of human dendritic cell precursors into IL12-producing DCs that elicit Th1-related cytokine production. In a first step, precursor DCs, obtained by treating purified monocytes with GMCSF and IL4 were incubated with JgD or JH. As shown in FIG. 6A, monocytes changed from individual and round cells to clumped cells with dendritic extensions after treatment with either JgD or JH. The immunophenotype of the cells (FIG. 6B) also changed with an upregulation of CD86 and HLA-DR expression within 72 h of treatment. Similar results were obtained for DC precursors treated with JH. The morphology, behavior, and increased expression of CD86 and HLA-DR are consistent with maturation of the DC precursors to mature DCs.

Different types of DCs are characterized by the cytokines that they produce and the subsequent T cell responses that they mediate. A survey of cytokine production was performed by protein array to determine the nature of the DC that was produced upon treatment of the DC precursors with JgD or JH. The protein array analysis is a very sensitive assay for the presence of multiple cytokines giving an output similar to a western blot. Densitometric values of spots indicate the amount of cytokine present in the spent medium of cells from treated or untreated cells. The normalized ratio of values for the cytokines in spent medium from treated or untreated cells provides a semi-quantitative analysis of the cytokine spectrum produced by the cells. Treatment with either the unconjugated H or gD epitopes or the J-ICBL caused no significant production of cytokines.

FIG. 7 shows those cytokines whose production was enhanced after a 72 h treatment with JgD or JH. The amount of IL12p70 was significantly increased by >4-fold following either treatment compared to control (Per ANOVA, p-values=2.03×10⁻⁵ (JgD) and 3.31×10⁻⁵ (JH) when compared to normalized untreated IL12p70 values) with a visible change in the levels of MCP-2 and RANTES. These results were reproduced for three different individuals and were similar following treatment with either JgD or JH.

In each case, IL12p70 production was enhanced following treatment with either JgD or JH but production of IL1, TNFα and IL6 was the same as untreated cells. Production of IL12p70 without concomitant enhancement of these proinflammatory cytokines is a different outcome than obtained with treatment by two TLR ligands, such as LPS and CpG.

Tests with mouse bone marrow cells showed that JgD or JH treatment was sufficient to convert DC precursor cells into IL12p70 producing DCs without a need for the addition of other cytokines or TLR ligands. Similarly, JgD treatment of human monocytes was sufficient to promote the maturation of these cells into DCs that produce IL12p70.

Table 2 shows the cytokine protein array ratios for monocytes, monocytes treated with GMCSF (G-monocytes); or, DC precursors generated with GMCSF plus IL4 (GM-4 monocytes). The levels of IL12p70, RANTES, MCP-1, and MCP-2 produced by monocytes after 24 h treatment with only JgD was most similar to cells pretreated for 24 h with GMCSF and then JgD. The GM-4 monocytes also produced elevated levels of IL12p70 and MCP-2 but the trends for some other cytokines and chemokines differed from that of JgD treated monocytes or GM-monocytes.

Ultimately, a DC1 cell must be able to activate T cells and promote IFNγ and IL2 production in order to mediate a Th1 immune response. The ability of JgD treated monocytes to support allotypic activation of T cells was tested. For the experiment depicted in FIG. 8, monocytes from two separate donors were treated with JgD or medium for 24 h prior to addition of T cells from other donors, and after 6 days spent medium was analyzed for cytokine production. Significantly large differences in the Th1 cytokines, IFNγ and IL2, were present in the spent medium from T cells mixed with JgD treated monocytes compared to those mixed with untreated monocytes. The same results were obtained with monocytes and T cells from another set of donors. No changes in cytokine production followed JgD treatment of a T cell-containing lymphocyte pool purified by elutriation and cell sorting (based on light scatter parameters) (data not shown). These results demonstrate that the JgD acts on monocytes to promote their maturation into DCs capable of promoting a Th1-like cytokine response by T cells.

### Discussion of Example 2

Conjugation of an antigenic peptide to the J-ICBL appears to create an immunogen that can activate and promote the maturation of dendritic cell precursors into DCs which produce IL12p70. Treatment of mouse bone marrow cells with JgD or JH, but not the unconjugated J-ICBL or gD or H peptides, promoted the maturation of DC precursors from bone marrow into IL12p70 producing DCs. The cells generated by treatment with JgD could present antigen to immune T cells to generate a booster-like enhancement of IFNγ production. In a similar manner, monocytes, GM-monocytes and GM-4 monocytes treated with either JgD or JH, produced cells which phenotypically resemble DCs and produce IL12p70, whereas GM-4 monocytes treated with the unconjugated gD, H or J peptides did not.

Although very similar, the cytokine/chemokine profile produced by JgD treated GM-4 monocytes differed from that of JgD treated monocytes or monocytes treated with GMCSF. Interestingly, JgD treatment of monocytes or GM-monocytes did not generate the proinflammatory cytokines TNFα, IL1 or IL6 but very small amounts of these cytokines were produced after JgD treatment of GM-4 monocytes. This shows that the type of DC generated by JgD treatment depends upon the nature of the starting cell. The IL4 treated monocyte behaves differently and differentiates into a different IL12-producing DC after JgD treatment than monocytes or GM-monocytes.

The DCs generated by JgD treatment were sufficient to promote Th1-like cytokine responses upon allotypic interactions with T cells. While this may not definitively demonstrate antigen specificity, it does demonstrate that sufficient amounts of IL12p70 are generated by the JgD-DCs to steer the cytokine response of the T cells with which they interact towards a Th1 response, which is characterized by the production of IFNγ and IL2.

Addition of JgD to monocytes was sufficient to convert the cells into a unique type of IL12-producing DC. Unlike DCs generated with multiple other TLR ligands, such as Lipid A or MPL, LPS, CpG, DNA or RNA, these cells did not produce increased amounts of IL1, TNFα, or IL6. The mechanism of induction promoted by JgD or JH is likely to be the result of cross-linking of the receptor for the J-ICBL to MHC I molecules through the linked epitope within the heteroconjugate peptide. This complex is likely to remain on the cell surface for long periods since mouse DCs bearing JgD could be washed free of unbound peptide after 24 h incubation and still provide an antigenic boost to immune splenic T cells.

Thus, the J-LEAPS immunogens, exemplified by JgD and JH, are sufficient to convert monocytes to a unique form of DC that produces IL12 but not acute phase cytokines and is sufficient to activate Th1 responses.

### Example 3

Herpes Simplex Virus Challenge in the Zosteriform Spread Mouse Model

Mouse bone marrow cells treated with JgD, incubated for 24 h, washed free of unbound vaccine or media components and injected subcutaneously or intraperitoneally initiated protection from disease and death from lethal herpes simplex virus challenge in the zosteriform spread mouse model.

Mice (C57BL/6) received two injections of either JgD-DC or untreated bone marrow cells. JgD-DC were prepared by treating bone marrow cells with JgD for 24 h and the cells were washed free of peptide and media components. JgD-DC or bone marrow cells were injected intradermally and intraperitoneally with a two week window and then received a lethal challenge with HSV-1 H129 in the zosteriform-challenge model. Mice were either untreated, treated with 24 h cell cultured bone marrow cells (BM), J-ICBL treated bone marrow cells (J-BM), JH treated bone marrow cells (JH-DC), or JgD treated bone marrow cells (JgD-DC). (0: no disease; 1: non-specific changes; 2: local disease; 3: early zosteriform spread; 4: later zosteriform spread with sores; 5: moribund disease; 6: death). Mice were scored daily for symptoms and the average for the group is presented.

In contrast, mice receiving no treatment, untreated mouse bone marrow cells (BM), mouse bone marrow cells treated with the J immune cell binding ligand only (J-BM), and/or mouse bone marrow cells treated with JH JH-DC), incurred significant disease with zosteriform spread and death of a majority of the group within 2 weeks. Whereas, all of the mice receiving bone marrow cells treated with JgD (JgD-DC) or bone marrow cells and challenged 1 with HSV-survived and most showed n signs of disease (6 of 7).

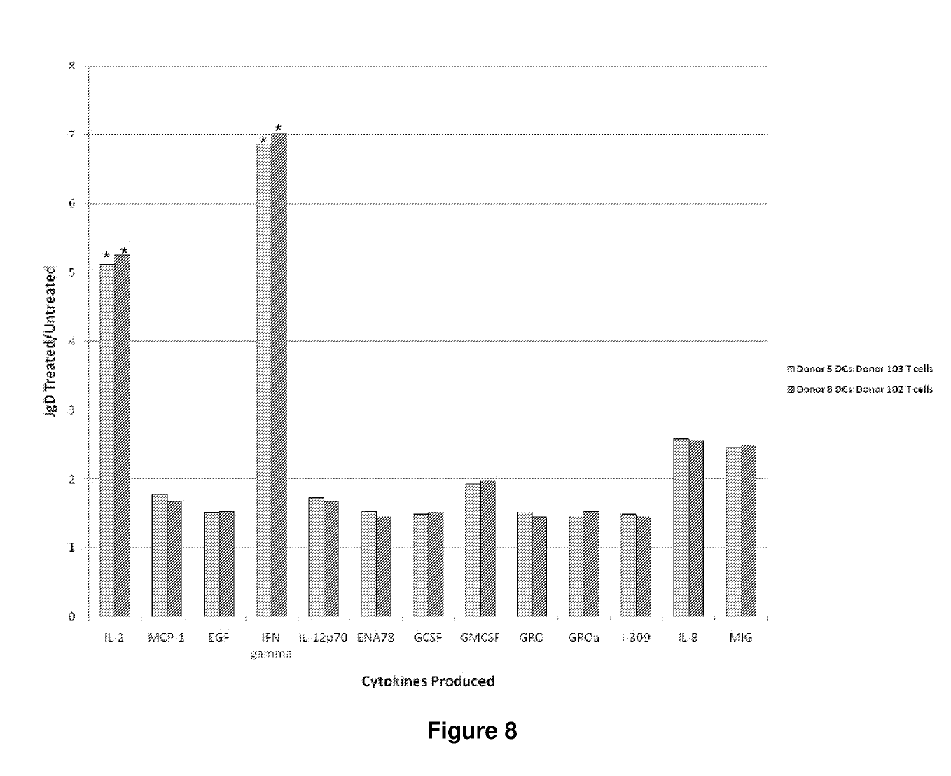

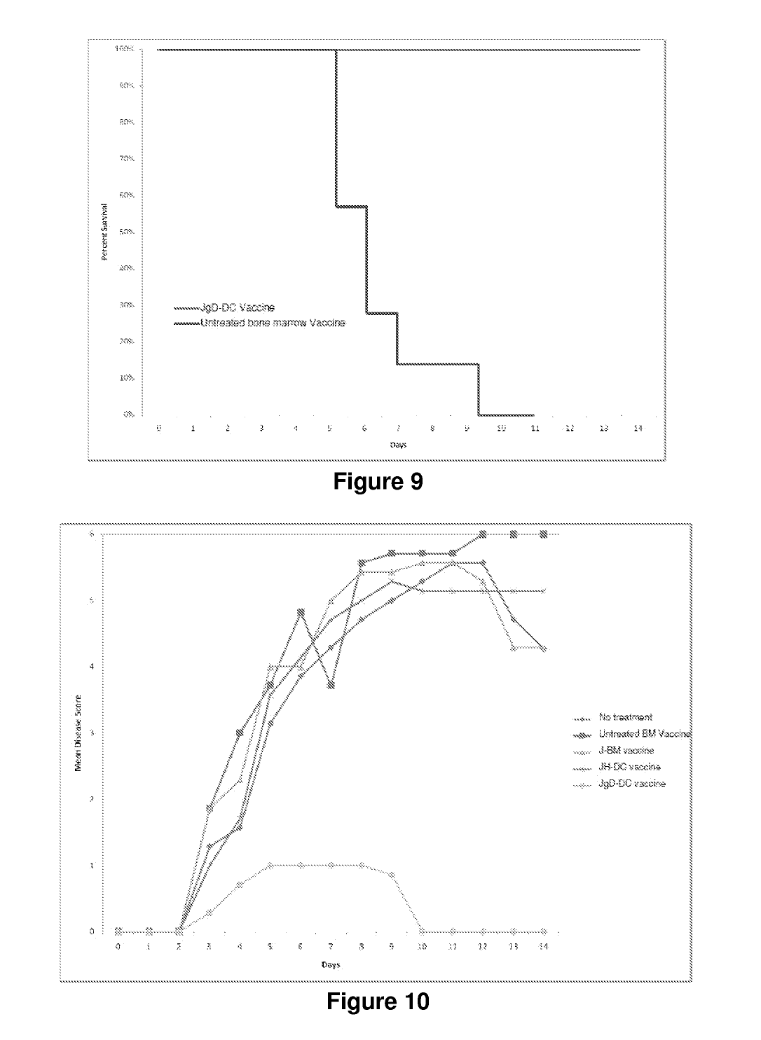

FIG. 9 shows a Kaplan Meier survival curve for the JgD-DC and untreated BM vaccinated mice. FIG. 10 is a disease score plot showing a reduction in prevention of symptoms of disease signs for mice treated with JgD-DC vaccine, as compared with: No treatment; Untreated BM vaccine; J-BM vaccine; and JH-DC vaccine.

These results prove that the DCs generated by JgD treatment of bone marrow cells is sufficient to initiate and develop an immune response sufficient to provide protection from a large lethal HSV infection.

These results prove that the LEAPS peptide stays on the surface of the DC for long periods and can interact with T cells to elicit the response.

### Example 4

Rheumatoid Arthritis

Disease signs consistent with rheumatoid arthritic disease progression were stopped by a LEAPS peptide conjugate P₁-x-P₂ in which P₁ is “J” [SEQ ID NO:1] and P₂ is a peptide from human type II collagen. Mice were treated to induce autoimmunity to collagen and develop disease signs consistent with rheumatoid arthritis.

The LEAPS peptide conjugate administered after the development of disease stopped disease progression at least as well if not better than etanercept (Enbrel), the drug of choice for late stage disease, which is a receptor antagonist and blocks the action of tumor necrosis factor alpha. The LEAPS peptide conjugate therapy as a vaccine in adjuvant was administered several times over a 90 period and was well tolerated and effective.

It is to be understood that, in certain embodiments, a preferred method of use can be iDCs of the patient mixed and incubated with the J L.E.A.P.S.™ conjugate before administration into the patient.

It is to be understood that, in certain embodiments, a preferred method of use can be iDCs of the patient mixed and incubated with the J L.E.A.P.S.™ conjugate and patient T cells before administration into the patient.

### Example 5

Exemplary Uses

In particular, the “DC-(P₁-x-P₂)” complex can provide the following pharmacological effects upon administration to a subject: suppression of inflammation, hypersensitivity and irritation; direct antiviral action against a broad range of pathogenic viruses, and palliative effects on inflammation or irritation caused by the viral infection.

Compositions may be suitable formulated as a pharmaceutical composition for topical, transdermal, intradermal or parenteral administration.

Thus, embodiments according to the invention such as i) compositions comprising the “DC-(P₁-x-P₂)” complex, ii) use of the “DC-(P₁-x-P₂)” complex for preparation of a medicament for immunomodulation in a mammal, or iii) a method for immunomodulation comprising administering the “DC-(P₁-x-P₂)” complex relate to one or more of the following diseases, disorders or conditions that involves immunomodulation:

Hypersensitivity and/or Inflammatory Reactions.

According to the invention all known conditions and diseases associated with inflammation and hypersensitivity reactions are relevant including I-IV type hypersensitivity and those caused by direct histamine release, and the following examples are not limiting with respect to this: infections (viral, bacterial, fungal, parasitic, etc.), cold and flu, contact dermatitis, insect bites, allergic vasculitis, postoperative reactions, transplantation rejection (graft-versus-host disease), asthma, eczema (e.g. atopic dermatitis), urticaria, allergic rhinitis, anaphylaxis, autoimmune hepatitis, Primary biliary cirrhosis, Primary sclerosing cholangitis, Autoimmune hemolytic anemias, Grave's disease, Myasthenia gravis, Type 1 Diabetes Mellitus, Inflammatory myopathies, Multiple sclerosis, Hashimoto's thyreoiditis, Autoimmune adrenalitis, Crohn's Disease, Ulcerative Colitis, Glomerulonephritis, Progressive Systemic Sclerosis (Scleroderma), Sjogren's Disease, Lupus Erythematosus, Primary vasculitis, Rheumatoid Arthritis, Juvenile Arthritis, Mixed Connective Tissue Disease, Psoriasis, Pemphigus, Pemphigoid, Dermatitis Herpetiformis, etc.

Inflammation and/or Hypersensitivity of the Skin, Such as Dermis and Mucous.

This effect can be obtained in relation to any skin disease or in relation to any disease that causes such symptoms of the skin Examples of such conditions are but not limited to atopic eczema, contact dermatitis, seborrhoeic eczema, infections and/or psoriasis.

Allergic Reactions and Conditions.

The therapeutic action may be relevant to allergic reactions and conditions, and the following examples are not limiting with respect to this: asthma, eczema (e.g. atopic dermatitis), urticaria, allergic rhinitis, anaphylaxis, etc.

Autoimmune Diseases and/or Chronic Inflammatory Diseases.

The therapeutic action may be relevant to all known autoimmune disorders, and the following examples are not limiting with respect to this: Autoimmune hepatitis, Primary biliary cirrhosis, Primary sclerosing cholangitis, Autoimmune hemolytic anemias, Grave's disease, Myasthenia gravis, Type 1 Diabetes Mellitus, Inflammatory myopathies, Multiple sclerosis, Hashimoto's thyreoiditis, Autoimmune adrenalitis, Crohn's Disease, Ulcerative Colitis, Glomerulonephritis, Progressive Systemic Sclerosis (Scleroderma), Sjogren's Disease, Lupus Erythematosus, Primary vasculitis, Rheumatoid Arthritis, Juvenile Arthritis, Mixed Connective Tissue Disease, Psoriasis, Pemfigus, Pemfigoid, Dermatitis Herpetiformis, etc.

Other Therapeutic Areas

In addition to specific therapeutic areas, the action of the “DC-(P₁-x-P₂)” complex is relevant to all known conditions and diseases associated with hypersensitivity reaction, and the following examples are not limiting with respect to this: infections (viral, bacterial, fungal, parasitic, etc.), cold and flu, contact dermatitis, insect bites, allergic vasculitis, postoperative reactions, transplantation rejection (graft-versus-host disease), etc. associated with inflammation or irritation in the respiratory system; prostatitis or benign prostatic hypertrophy inflammation of various tissues, e.g. inflammation of the prostate, in particular prostatitis; cardiovascular disease, especially hyperlipidemia and atherosclerosis; cancer; alleviation of pain.

Viral Infections

In interesting embodiments according to the invention, the method of treating relates to viral infections such as those caused by various types of herpes simplex or other viruses as discussed herein.

Non-limiting examples of families of viruses are the herpes viruses such as Herpes simplex virus (HSV) including HSV-1 which causes herpes labialis (cold sore), herpetic stomatitis, keratoconjunctivitis and encephalitis, and HSV-2 which causes genital herpes and may also be responsible for systemic infection. Another member of the herpes virus family is Varicella zoster virus (VZV). VZV causes two distinct diseases: varicella (chickenpox) and herpes zoster (shingles). Yet another member of the herpes virus family is cytomegalovirus (CMV). Another member of the herpes virus family is Epstein-Ban virus (EBV). Yet another member of the herpes virus family is human herpes virus type 6 (HHV-6).

Other families of viruses include, for example: the adenoviruses; the papovaviruses, such as human papillomavirus (HPV), those implicated in the etiology of carcinoma of the cervix (types 16 and 18), BK virus (a polyomavirus), etc.; the parvoviruses, such as the parvovirus which produces erythema infectiosum (fifth disease); the picornaviruses, such as polioviruses, coxsackievirus, echovirus and enterovirus, rhinoviruses; the reoviruses, such as rotavirus; the togaviruses, such as rubella virus, arbovirus, flaviviruses such as Yellow fever and dengue fever; the bunyaviruses, such as haemorrhagic fever viruses, hantavirus; the orthomyxoviruses, such as influenza A (such as the sub-types H1Ni, Spanish flu, Avian flu, Swine flu and the like), influenza B and influenza C; the paramyxoviruses, such as measles (rubella), mumps; the Rhabdoviruses, such as rabies virus; the retroviruses, such as HIV-1 and HIV-2 (the cause of AIDS); the arenaviruses, such as lymphocytic choriomeningitis, lassa fever; Marburg virus disease and Ebola virus disease.

Vaccines

The DC-conjugated peptide complexes may be used as a vaccine either prophylactically or therapeutically. When provided prophylactically the vaccine is provided in advance of any evidence of disease. The prophylactic administration of the invention vaccine can serve to prevent or attenuate disease in a subject. In one preferred embodiment, a human, at high risk for a disease is prophylactically treated with a vaccine of this invention. When provided therapeutically, the vaccine is provided to enhance or modulate the patient's own immune response and, hence, control of disease.

For example, in the case of autoimmune diseases, asthma, allergy, and transplantation rejection, the desired outcome is the inhibition/suppression, rather than the stimulation/activation, of the immune response, in an antigen-specific manner. This desired outcome is due to the fact that antigen-specific response by T cells and also B cells may, in many instances, lead to an undesirable immune response outcome, culminating in autoimmune disease (in the case of autoantigens), asthma or allergy (in the case of allergens) and transplantation rejection (in the case of transplantation antigens). The ability to markedly decrease or completely retard, in an antigen specific manner, undesirable immune response outcomes, while maintaining the remainder of the immune response intact, is achieved through the DC-conjugated peptide construct complexes described herein.

The DC-conjugated peptide complexes of this invention may be used as therapeutic compounds for the treatment of autoimmune diseases and conditions, and for treatment of allergy and asthma and transplantation rejection in humans and other animals, preferably mammals, including household pets, such as dogs and cats, as well as livestock, such as bovine, porcine and equine. The DC-conjugated peptide complexes may also be used prophylactically in humans and other animals to inhibit the likelihood of onset of autoimmune disease, allergy or asthma in individuals considered to be at risk for such conditions, whether as a result of genetic factors or environmental exposure, age or other factors.

The DC-conjugated peptide complexes may be administered alone (in a suitable vehicle depending on the mode of administration) or in combination or in conjunction with an adjuvant or other active component, including, for example, any conventional treatment therapy for the particular condition to be treated.

Preparations containing the subject peptide constructs may be administered by any of the known methods for peptide administration, including, for example, intramuscularly (IM), subcutaneously (SC), transdermally, or intranasally or orally, or as an inhalant preparation or intravenously. These preparations may be formulated as unit dosages to provide a therapeutically effective amount of the conjugated peptide, preferably an amount in the range of 10 to 100 micrograms per kilogram of body weight. Usually, the therapeutic or prophylactic preparations will be administered over a prolonged course of administration, such as weekly, bi-weekly, monthly, quarterly, semi-annually or annually, often for a patient's lifetime. The prolonged treatment will generally be necessary since newly formed or mature T cells with the antigen-specific TCR of interest, can be expected to be produced by the bone marrow and re-enter into the blood and lymphatic system, even after the initial treatment, over the course of an individual's lifetime.

The DC-conjugated peptide complexes of this invention are also useful in connection with prevention or inhibition of transplantation rejection in animals (humans and other mammals) undergoing tissue or organ transplantation. Such transplantation rejection may take the form of host-versus-graft (HvG) rejection or as graft-versus-host (GvH) rejection, the latter being especially severe in immunocompromised and severely immunosuppressed individuals.

In the case of HvG, the host immune response cells, T cells, B cells, and macrophages, are activated by donor antigens (e.g., HLA antigens and other non-HLA antigens) that are specific for the donor cells and which the host perceives as “foreign.” The host immune cells attack the donor organ resulting in graft rejection.

In the case of GvH, the donor cells (especially as a result of bone marrow transplantation) respond to the host's cells/organs(s) as foreign antigens resulting in cellular infiltration of the host's organs, culminating in multiple organ failure, and often, death.

For treating transplantation rejection in the case of organ donation, i.e., HvG, the host may be injected with from about 10 to about 100 micrograms per kilogram of body weight with peptide construct(s) using as P₁ unique antigen(s) of the donor specific organ antigen, or preferably, a mixture of different donor specific antigens P₁.

Dosage amounts and modes of administration are similar to the dosages and modes of administration for GvH, namely, for example, about 10 to 100 micrograms/kilogram body weight, via intravenous infusion, every other day for 2 to 3 weeks, and then monthly, bi-monthly, semi-annually or annually, thereafter, in the recipient following organ transplantation. This treatment will result in depletion of the recipient's immune T cells which would otherwise be available to react with donor organ antigens, leading to the inhibition of host-vs-graft rejection.

When provided prophylactically the vaccine can be provided in advance of any evidence of disease. The prophylactic administration of the vaccine should serve to prevent or attenuate the disease in a mammal. In a preferred embodiment a human, at high risk for such disease can be prophylactically treated with a vaccine of this invention.

When provided therapeutically, the vaccine is provided to enhance the patient's own immune response to the disease antigen and, hence, control of disease.

Formulations

While it is possible for the immunogenic DC-conjugated peptide complexes to be administered in a pure or substantially pure form, it is preferable to present it as a pharmaceutical composition, formulation or preparation.

The formulations of the present invention, both for clinical and for human use, comprise a DC-conjugated peptide complex as described above, together with one or more pharmaceutically acceptable carriers and, optionally, other therapeutic ingredients. The carrier(s) must be “acceptable” in the sense of being compatible with the other ingredients of the formulation and not deleterious to the recipient thereof. The formulations may conveniently be presented in unit dosage form and may be prepared by any method well-known in the pharmaceutical art.

In general, the formulations are prepared by uniformly and intimately bringing into association the active ingredient with liquid carriers or finely divided solid carriers or both, and then, if necessary, shaping the product into the desired formulation.

Formulations suitable for any route of administration may be used, such as, for example, intravenous, intramuscular, subcutaneous, intraperitoneal, nasal, oral, rectal, vaginal, etc. Generally, the formulations will comprise sterile aqueous solutions of the active ingredient with solutions which are preferably isotonic with the blood of the recipient. Such formulations may be conveniently prepared by dissolving solid active ingredient in water containing physiologically compatible substances such as sodium chloride (e.g. 0.1-2.0M), glycine, and the like, and having a buffered pH compatible with physiological conditions to produce an aqueous solution, and rendering the solution sterile. These may be present in unit or multi-dose containers, for example, sealed ampoules or vials.

The formulations of the present invention may incorporate a stabilizer. Illustrative stabilizers include polyethylene glycol, proteins, saccharides, amino acids, inorganic acids, and organic acids which may be used either on their own or as admixtures. These stabilizers, when used, are preferably incorporated in an amount of about 0.1 to about 10,000 parts by weight per part by weight of immunogen. If two or more stabilizers are to be used, their total amount is preferably within the range specified above. These stabilizers are used in aqueous solutions at the appropriate concentration and pH. The specific osmotic pressure of such aqueous solutions is generally in the range of about 0.1 to about 3.0 osmoles, preferably in the range of about 0.3 to about 1.2. The pH of the aqueous solution is adjusted to be within the range of about 5.0 to about 9.0, preferably within the range of 6-8. In formulating the immunogen of the present invention, anti-adsorption agent may be used.

Additional pharmaceutical methods may be employed to control the duration of action. Controlled release preparations may be achieved through the use of polymer to complex or absorb the conjugated polypeptide. The controlled delivery may be exercised by selecting appropriate macromolecules (for example polyester, polyamino acids, polyvinyl, pyrrolidone, ethylenevinylacetate, methylcellulose, carboxymethylcellulose, or protamine sulfate) and the concentration of macromolecules as well as the methods of incorporation in order to control release.

Another possible method to control the duration of action by controlled-release preparations is to incorporate the DC-conjugated peptide complexes into particles of a polymeric material such as polyesters, polyamino acids, hydrogels, poly(lactic acid) or ethylene vinylacetate copolymers. Alternatively, instead of incorporating these agents into polymeric particles, it is possible to entrap these materials in microcapsules prepared, for example, by coacervation techniques or by interfacial polymerization, for example, hydroxy-methylcellulose or gelatin-microcapsules and poly(methylmethacrylate) microcapsules, respectively, or in colloidal drug delivery systems, for example, liposomes, albumin microspheres, microemulsions, nanoparticles, and nanocapsules or in macroemulsions.

When oral preparations are desired, the compositions may be combined with typical carriers, such as lactose, sucrose, starch, talc, magnesium stearate, crystalline cellulose, methyl cellulose, carboxymethyl cellulose, glycerin, sodium alginate or gum arabic among others. These carriers may likewise be used for preparing to be administered via other cavities, e.g., nasal, rectal, etc.

Kits

The DC-conjugated peptide complexes of the present invention may be supplied in the form of a kit, alone, or in the form of a pharmaceutical composition as described above.

Vaccination can be conducted by conventional methods. For example, the immunogenic DC-conjugated peptide complex can be used in a suitable diluent such as saline or water, or complete or incomplete adjuvants. The immunogen can be administered by any route appropriate for antibody production such as intravenous, intraperitoneal, intramuscular, subcutaneous, and the like. The immunogen may be administered once or at periodic intervals until, for example, a significant titer of CD4⁺ or CD8⁺ T cell and/or antibodies directed against the antigen is obtained.

In particular, the antigenic polypeptides of the DC-conjugated peptide comples elicit TH1 associated antibodies and other aspects of a TH1 immune response. The presence of immune cells versus non-immune cells may be assessed in vitro by measuring cytokine secretion, lymphoproliferation, cell activation markers, cytotoxicity, or altered metabolism, in response to T cells pulsed with the immunogen or by DTH using the conjugated polypeptide in vivo. The antibody may be detected in the serum using conventional immunoassays.

As noted above, the administration of the vaccine of the present invention may be for either a prophylactic or therapeutic purpose. When provided prophylactically, the immunogen is provided in advance of any evidence or in advance of any symptom due to the disease, especially in patients at significant risk for occurrence. The prophylactic administration of the immunogen serves to prevent or attenuate the disease in a human.

When provided therapeutically, the immunogen is provided at (or after) the onset of the disease or at the onset of any symptom of the disease. The therapeutic administration of the immunogen serves to attenuate the disease.

Preparation of Conjugated Peptides

The conjugated polypeptides, which may be prepared by conventional solid phase peptide synthesis or other conventional means for peptide synthesis, however, the peptides may also be prepared by genetic engineering techniques. The DNA sequences coding for the peptides of this invention can be prepared by any of the well known techniques for recombinant gene technology. For example, reference can be made to the disclosure of recombinant proteins and peptides in U.S. Pat. No. 5,142,024 and the body of literature mentioned therein, the disclosures of which are incorporated herein by reference thereto.

Thus, this invention also provides a recombinant DNA molecule comprising all or part of the nucleic acid sequence encoding the antigenic peptide or the immunomodulatory peptide for subsequent direct linking or linking via a linking group, as previously described, or, more preferably, encoding the conjugated polypeptide of formula P₁-x-P₂, as described above, and a vector. Expression vectors suitable for use in the present invention comprise at least one expression control element operationally linked to the nucleic acid sequence. The expression control elements are inserted in the vector to control and regulate the expression of the nucleic acid sequence. Examples of expression control elements include, but are not limited to, lac system, operator and promoter regions of phage lambda, yeast promoters and promoters derived from polyoma, adenovirus, retrovirus or SV40.

Additional preferred or required operational elements include, but are not limited to, leader sequence, termination codons, polyadenylation signals and any other sequences necessary or preferred for the appropriate transcription and subsequent translation of the nucleic acid sequence in the host system. It will be understood by one skilled in the art that the correct combination of required or preferred expression control elements will depend on the host system chosen.

In the present invention, one preferred example is peptide J from β-2-microglobulin (β-2M 35-50) (Parham, et al., 1983, J Biol Chem. 258:6179; Zimmerman, et al.) Other related β-2M peptides include amino acid residues 24 to 58 and amino acid residues 58 to 84, as described more fully in U.S. Pat. No. 5,652,342.

Examples of other peptides which may be used may be found in commonly assigned U.S. Pat. No. 5,652,342, the disclosure of which is incorporated herein in its entirety by reference thereto.

Guidelines for selection of these or other suitable T cell binding peptides are discussed therein as well as in the Zimmerman, et al. article. Mention may be made of, for example, the molecules known as B7 (Freeman, et al., Science 262:909); B70 (Azuma, et al., 1993, Nature 366:76); GL1 (Hathcock, et al., 1993, Science 262:905); CD58 (Arulanandam, et al., 1993, Proc. Nat. Acad. Sci. 90:11613), CD40 (van Essen, et al., 1995, Nature 378:620); and ICAM-1 (Becker, et al., 1993, J. Immunol. 151:7224). Other useful immunogenic peptides as P₁ include, for example, MHC class 1α3 domain comprising a.a. residues 223-229 or 223-230 (Peptide E, Salter, et al., Nature, 345:41, 1990); Interleuken Iβ, residues 163-171 (Nenconi, et al., J. Immunol. 139:800, 1987); MHC class IIβ2 domain, a.a. 135-149 (Konig, et al., Nature 356:796, 1992); Cammarota, et al., Nature 356:799, 1992). The reader is referred to these literature articles for further details.

Linking Groups

Conjugated polypeptides may be prepared by directly bonding an antigenic specific peptide P₂to an ICBL binding peptide P₁; or by bonding the peptides P₁ and P₂ via a linking group, by conventional techniques, as more particularly described in detail in the aforementioned U.S. Pat. No. 5,652,342, the disclosure of which is incorporated herein in its entirety by reference thereto. When x represents the divalent linking group, it may be comprised of one or more amino acids, such as, for example, glycine-glycine, or a bifunctional chemical linking group, such as, for example, N-succinimidyl-3-(2-pyridyldithio)propionate (SPDP), m-maleimidobenzoyl-N-hydroxy-succimide ester (MBS), 1-ethyl-3-(3-dimethylaminopropyl)carbodiimide (EDC), or any other reagent commonly employed to link peptides. Again, reference is made to the disclosure of U.S. Pat. No. 5,652,342 for further details.

Also, while the peptide P₁ and peptide P₂ may be directly coupled to each other, (i.e., x is a direct peptide bond) in some cases a small linker sequence or a larger heterolinker molecule may be advantageously used to couple the two peptides. For example, as the spacer, one or a few, up to about 5, preferably, up to about 3, neutral amino acids, such as glycine, may be used to link the peptides. A preferred spacer peptide is GGG, however, the spacer may be made larger or smaller and altered to include other molecules besides the amino acid glycine. As examples of heterolinkers mention may be made of, for example, N-succinimidyl-3-(2-pyridylthio)propinate (SPDP), m-maleimidobenzoyl-N-hydroxy-succimide (MBS) as well as any of the other reagents employed to link peptides, including without limitation those disclosed in the aforementioned U.S. Pat. No. 5,652,342. When the peptides P₁ and P₂ are not directly bonded the linking group will generally and preferably be any divalent linking group. The linking group may be cleavable or non-cleavable under physiological conditions or by appropriate inducement.

Although the total number of amino acids in the conjugated polypeptide is not particularly critical, from a practical aspect, the minimum number of amino acids, including any amino acid spacers or linkers, will generally be at least about 15 or 16, preferably at least about 20, to obtain adequate antigen presentation and immunogenicity. Moreover, from practical considerations of ease of manufacture by synthetic techniques, the maximum number of amino acids will often be less than about 100, preferably, no more than about 70, especially, no more than about 50. However, where the conjugated polypeptide may be manufactured by genetic engineering techniques, much larger molecules may be useful.

The linking group will generally be non-cleavable under the conditions of use, however, cleavable groups may also be used where it is desired to separate peptide P₁ or peptide P₂ after the conjugated peptide bonds to its target cell or T cell receptor on appropriate T cell. For example, the linking group x may be one which is enzymatically cleavable or cleavage may be induced, such as by photoactivation, including for example, exposure to UV radiation.

The immunogenic conjugated peptides of this invention can elicit an immune response that can be directed toward the desired TH1 as evidenced by the numerous examples of the TH1 characteristic antibody IgG2a (mouse) or IgG3 (man), and/or by a DTH response.

Administration

When used as a vaccine in the method of this invention, the vaccine can be introduced into the host most conveniently by injection, intramuscularly, intradermally, parenterally, orally or subcutaneously. Any of the common liquid or solid vehicles may be employed, which are acceptable to the host and which do not have any adverse side effects on the host or any detrimental effects on the vaccine. Phosphate buffered saline (PBS), at physiological pH, e.g. pH 6.8 to 7.4, preferably pH 7, may be used as a carrier, alone or with a suitable adjuvant. The concentration of immunogenic polypeptide may vary from about 0.1 to 200 μg/kg, such as about 25 μg/kg per injection, in a volume of clinical solvent generally from about 0.1 to 1 ml, such as about 0.2 ml, preclinical studies in animals, and from about 0.5 ml to about 2 ml, such as about 1 ml in humans. Multiple injections may be required after the initial injections and may be given at intervals of from about 2 to 4 weeks, for example, about 2 weeks in animals and about 8 weeks in humans, when multiple injections are given.

A preferred concentration of immunogenic polypeptide in the vaccines of the present invention may be in the range of from 10 to 25 μg/kg; however, a higher dose may be administered as needed.

### Example 6

Modifications of P₁-x-P₂ L.E.A.P.S.™ Constructs

The following are non-limiting examples of types of molecular modifications to the antigen presenting molecule, (also referred to as Peptide P₂) which will result in the blockade or inhibition of a second signal to the antigen-specific T cell clones as described above:

1. Single or few amino acid deletion(s);

2. Single or few amino acid substitution(s) and/or addition(s);

3. Disulfide bond formation at specific site(s) in the antigen presenting molecule;

4. Combination of any and all of the changes listed in 1, 2, and 3 above;

5. An amino acid sequence (R) of at least 4 amino acids, preferably at least 6 amino acids, more preferably at least about 8 or 9 amino acids, such as from about 10 to about 50 amino acids, and wherein “R” will not bind to the antigen of interest, herein P₁, and will not interact with the T cell accessory molecule(s) in such a way that would cause T cell activation when the TCR is engaged by P₂.

The specific amino acid modifications (deletions, additions and/or substitutions) in points 1, 2 and 3 above, are selected on the premise that homologous regions, in these molecules, are those most important for the overall functional integrity of these molecules. Thus, a comparison of, for example, the molecular protein structure of the HLA Class I and Class II molecular fragments derived from the intact molecules, among different species, have revealed different domains, within the structure of these molecules, that share molecular motifs. Similar observations apply to the other source molecules identified above, or any other source molecules.

For amino acid additions and substitutions, one or more than one of the conserved amino acids can be replaced by one or more amino acids. When a conserved amino acid is replaced by more than one amino acid, the replacement amino acids (preferably no more than about 15, preferably no more than about 10, especially, no more than about 5 or 6, such as 2 or 3) may be inserted in the amino acid sequence. The amino acids substitutions may also be added as side chain attachments bonded to, or replacing, one of the conserved amino acids. While the specific sequence of the added internal or side chain replacement amino acids is not particularly critical, care should be taken to select a sequence which will not bind or interact with the sequence P₂ and will not interact with the T cell accessory molecule(s) on the particular set or subset of T cells bearing the antigen specific TCR for P₂ to inadvertently cause T cell activation when the TCR is engaged by P₂.

For any given peptide the skilled practitioner will be able to determine suitable sequences for amino acid substitutions and/or additions. Usually, however, it should be sufficient to simply delete or replace one or more of the conserved (homologous) amino acids from the ICBL sequence. When replacing the conserved amino acid with a single amino acid it is generally preferred to select an amino acid having diverse properties and/or molecular size from as that of the conserved amino acid being replaced. For example, an acidic amino acid may be replaced with a basic amino acid. Other types of “non-conservative” types of amino acid substitutions are well known to the skilled practitioner.

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The invention also concerns a method for treating or preventing disease by administering to a human patient in need thereof a therapeutically effective amount of the conjugated polypeptide of this invention

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof. Therefore, it is intended that the invention not be limited to the particular embodiment disclosed herein contemplated for carrying out this invention, but that the invention will include all embodiments falling within the scope of the claims.

1. A complex, comprising:
a mature dendritic cell (DC) population capable of having an immunomodulatory effect, wherein at least one mature dendritic cell has at least one peptide construct at least partially attached or bound to the surface of the dendritic cell, the peptide construct having a formula: P₁-x-P₂, where
P₂ represents an antigenic peptide;
P₁ represents an immunomodulatory peptide which is a portion of an immunoprotein capable of promoting binding to a class or subclass of DC or T cells and which is capable of directing a subsequent immune response to the peptide P₂ to a Th1 or other immune response; and
x represents a covalent bond or a divalent peptide linking group, which may be cleavable or non-cleavable.

2. The complex of claim 1, wherein the dendritic cell (DC) population is matured with an effective amount of the peptide construct under conditions suitable for maturation of precursors of dendritic cells (iDCs) to form the mature dendritic cells (DCs).

3. The complex of claim 1, wherein the peptide construct is capable of modifying cellular and/or humoral immune responses of a subject by reacting with the subject's own immune system and/or cells derived from the subject's immune system, without need for adjuvants or “non-self” antigens.

4. The complex of claim 1, wherein the P₁x-P₂ construct generates a population of the mature dendritic cells (DCs) capable of producing interleukin 12 (IL-12) as compared to an iDC population not contacted with the peptide construct.

5. (canceled)

6. (canceled)

7. (canceled)

8. (canceled)

9. The complex of claim 1, wherein precursor, or immature, dendritic cells (iDCs) are derived from the subject.

10. The complex of claim 1, wherein the precursor, or immature, dendritic cells (iDCs) are derived from a donor compatible with the subject.

11. The complex of claim 1, wherein the Pₛ is an antigenic peptide, or fragment thereof, associated with a disease selected from one or more of: a cancer, an allergen, an autoimmune-related antigen, a transplantation autoimmune response, a tumor antigen, an acute, latent-recurring and/or chronic inflammatory response.

12. The complex of claim 11, wherein a causative agent of the disease to which the antigenic peptide is associated is one or more of: bacteria, viruses, fungi, protozoa, parasites and prions.

13. (canceled)

14. (canceled)

15. (canceled)

16. The complex of claim 1, wherein the complex promotes a systemic modulation of immune and inflammatory responses in the subject sufficient to initiate a non-specific immunomodulatory therapeutic response to a chronic condition in the subject.

17. (canceled)

18. (canceled)

19. (canceled)

20. (canceled)

21. The complex of claim 1, wherein the peptide construct comprises an immune cell binding ligand, termed “J”, an amino acid 38-50 from the β-2-microglobulin (DLLKNGERIEKVE) [SEQ ID NO:1], optionally conjugated to a peptide from the N-terminus of HSV-1 glycoprotein “D” (SLKMADPNRFRGKDLP) [SEQ ID NO:2], amino acid 8-23) through a triglycine linker.

22. The complex of claim 1, wherein the peptide construct comprises an immune cell binding ligand, termed “J”, an amino acid 38-50 from the β-2-microglobulin (DLLKNGERIEKVE) [SEQ ID NO:1], optionally conjugated to a HGP-30 peptide from the p17 HIV gag protein “H” (YSVHQRIDVKDTKEALEKIEEEQNKSKKKA) (aa 85-115)) [SEQ ID NO:3] through a triglycine linker.

23. A method for producing a mature dendritic cell (DC) population, comprising the step of:
contacting precursor, or immature, dendritic cells (iDCs) with an effective amount of a peptide construct under conditions suitable for forming mature dendritic cells (DC),
the peptide construct having a formula: P₁-x-P₂, where
P₂ represents a specific antigenic peptide;
P₁ represents an immunomodulatory peptide which is a portion of an immunoprotein capable of promoting binding to a class or subclass of DC or T cells and which is capable of directing a subsequent immune response to the peptide P₂ to a Th1 or other immune response; and
x represents a covalent bond or a divalent peptide linking group, which may be cleavable or non-cleavable.

24. The method of claim 23, wherein the population of mature DCs produces an immunomodulating response with an increased amount of interleukin 12 (IL-12) as compared to an iDC population not contacted with the peptide construct.

25. The method of claim 23, wherein the precursor, or immature, dendritic cells (iDCs) comprise one or more of: blood derived monocytes and bone marrow cells.

26. The method of claim 23, wherein the peptide construct is capable of directly inducing a dendritic cell immune response, wherein dendritic cell maturation is increased.

27. The method of claim 23; wherein the mature DCs are characterized by up-regulation of at least one of: CD11c, CD86, MHC class I or MHC class II cell surface markers.

28. The method of claim 23; wherein the mature DCs are capable of producing a desired cytokine profile.

29. The method of claim 23, wherein the mature DCs produce interleukin 12 (IL-12).

30. A method of treating a subject in need thereof, comprising administering an effective amount of the mature DCs produced according to the method of claim 23 directly into or around a tumor, infected tissue or organ presented by the subject, or into a draining lymph node or peritoneum of the subject.

31. The method of claim 30, including inducing proliferation of a cell population containing mature dendritic cells (DCs) by contacting blood derived monocytes and/or bone marrow cells of the subject with the peptide construct.

32. The method of claim 30, wherein the subject is a human.

33. An autologous method for inducing and/or modulating a response to an immunogen in a subject in need thereof, comprising:
i) combining precursor, or immature, dendritic cells (iDCs) extracted from the subject with a peptide construct having the formula P₁-x-P₂ to form a complex, the peptide construct having a formula: P₁-x-P₂, where
P₂ represents a specific antigenic peptide;
P₁ represents an immunomodulatory peptide which is a portion of an immunoprotein capable of promoting binding to a class or subclass of DC or T cells and which is capable of directing a subsequent immune response to the peptide P₂ to a Th1 or other immune response; and
x represents a covalent bond or a divalent peptide linking group, which may be cleavable or non-cleavable; and
ii) administering the complex to the subject.

34. The method of the claim 33, wherein the mixture is administered to the subject after the mixing step without any further incubation of the iDCs.

35. The method of the claim 33, wherein the mixture is administered to the subject after ex vivo incubation of the iDCs in cell culture.

36. The method of claim 33, further comprising:
differentiating the precursor, or immature, dendritic cells (iDC) from the subject ex vivo into mature dendritic cells (DCs) in the presence of the peptide construct.

37. The method of claim 33, wherein the
precursor, or immature, dendritic cells (iDCs) are from blood derived monocytes and/or bone marrow taken from the subject.

38. (canceled)

39. (canceled)

40. An isolated mature dendritic cell (DC) population, comprising DCs capable of producing an immunomodulatory response, the mature DCs being prepared by maturation of precursor, or immature, dendritic cells (iDCs) in the presence of a peptide construct under conditions suitable for the maturation of the dendritic cells,
the peptide construct having a formula: P₁-x-P₂, where
P₂ represents a specific antigenic peptide;
P₁ represents an immunomodulatory peptide which is a portion of an immunoprotein capable of promoting binding to a class or subclass of DC or T cells and which is capable of directing a subsequent immune response to the peptide P₂ to a Th1 or other immune response; and
x represents a covalent bond or a divalent peptide linking group, which may be cleavable or non-cleavable.

41. A pharmaceutical composition comprising an effective amount of the complex of claim 1.

42.-65. (canceled)

66. A method for inducing a Th1 response in a subject suitable for the treatment of a cancer or an infectious disease, the method comprising the steps of:
i) exposing isolated immature dendritic cells to a P₁-x-P₂ peptide construct to form a DC-peptide conjugate mixture;
the peptide construct having a formula: P₁-x-P₂, where
P₂ represents a specific antigenic peptide;
P₁ represents an immunomodulatory peptide which is a portion of an immunoprotein capable of promoting binding to a class or subclass of DC or T cells and which is capable of directing a subsequent immune response to the peptide P₂ to a Th1 or other immune response; and
x represents a covalent bond or a divalent peptide linking group, which may be cleavable or non-cleavable; and
ii) removing free peptide construct from the mixture to form a complex, and
iii) administering the complex to a subject whereby the immune response generated in the subject is sufficient to prevent the onset or progression of cancer or to prevention infection with a pathogenic micro-organism and thereby prevent an infectious disease.

67. An anti-cancer vaccine complex comprising a peptide construct that binds to an immature dendritic cell.

68.-71. (canceled)

72. A method for activating T cells in a subject, comprising:
i) providing precursor, or immature, dendritic cells (iDCs);
ii) contacting the iDCs with at least one peptide construct during a time period sufficient for binding of the peptide construct to the iDCs;
the peptide construct having a formula: P₁-x-P₂, where
P₂ represents a specific antigenic peptide;
P₁ represents an immunomodulatory peptide which is a portion of an immunoprotein capable of promoting binding to a class or subclass of DC or T cells and which is capable of directing a subsequent immune response to the peptide P₂ to a Th1 or other immune response; and
x represents a covalent bond or a divalent peptide linking group, which may be cleavable or non-cleavable; and
iii) culturing under conditions suitable for maturation of the iDCs to form a mature dendritic cell (DC) population; and;
iv) contacting the mature DC population with T cells from the subject.

73. The method of claim 72, wherein the T cells and the iDCs are autologous to each other.

74.-78. (canceled)

79. A vaccine comprising the complex of claim 1.

80. The vaccine of claim 79, wherein the precursor, or immature, dendritic cells were originally isolated from the human subject

81. The vaccine of claim 79, wherein the peptide construct encodes a pathogen-specific antigen.

82. The vaccine of claim 81, wherein the pathogen-specific antigen comprises at least one antigen from: HIV, HSV and Influenza A virus.

83.-98. (canceled)

99. The method of claim 23, wherein DC precursor cells are incubated with the P₁-x-P₂ peptide constructs with one or more of: GM-CSF (granulocyte monocyte colony stimulating factor), and IL4 (interleukin 4).

100. The method of claim 23, wherein DC precursor cells are incubated with the P₁-x-P₂ peptide constructs without one or more of: GM-CSF (granulocyte monocyte colony stimulating factor), and IL4 (interleukin 4).

101. The vaccine of claim 79, wherein the peptide construct encodes a disease-specific antigen.

102. The vaccine of claim 79, wherein the disease-specific antigen comprises a peptide related to a tumor or an autoimmune disease.


SEQUENCE LISTING (3 sequences)

SEQ ID NO: 1 - protein, 13 aa, Homo sapiens
DLLKNGERIEKVE

SEQ ID NO: 2 - protein, 16 aa, Herpes simplex virus
SLKMADPNRFRGKDLP

SEQ ID NO: 3 - protein, 30 aa, Human immunodeficiency virus
YSVHQRIDVKDTKEALEKIEEEQNKSKKKA